(19) United States
(12) Patent Application Publication
(10) Pub. No.: US 2025/0163484 A1
(43) Pub. Date: May 22, 2025

(54) Methods for Incorporating Azido Groups in Bacterial Capsular Polysaccharides

(71) Applicant: PFIZER INC., New York, NY (US)

(72) Inventors: Ali GHOORCHIAN, Apex, NC (US); Samuel Isaac JENKINS, New Hill, NC (US); Nicholas Peter VITKO, Pittsboro, NC (US); Richard Jefferson WAYLAND, Raleigh, NC (US); Karen Leanza WILSON, Pittsboro, NC (US)

(21) Appl. No.: 18/839,636

(22) PCT Filed: Feb. 22, 2023
(86) PCT No.: PCT/IB2023/051620

§ 371 Date: Aug. 19, 2024

Related U.S. Application Data
(60) Provisional application No. 63/314,008, filed on Feb. 25, 2022.

(51) Int. Cl. C12P 19/26; A61K 39/09; C07H 1/06; C07H 5/04; C12R 1/46

(52) U.S. Cl. CPC C12P 19/26; A61K 39/092; C07H 1/06; C07H 5/04; C12R 2001/46

(57) ABSTRACT

The present invention relates to fermentation methods for incorporating azido groups in bacterial capsular polysaccharides. The invention also relates to capsular polysaccharides comprising azido groups obtained by said methods. The capsular polysaccharides comprising azido groups may be used for click-based conjugation with alkyne-labeled carrier to generate glycoconjugates. The polysaccharides and glycoconjugates obtained by the method may be further used for preparation of immunogenic compositions for preventing and treating infections caused by the organism.

### FIELD OF THE INVENTION

The present invention relates to fermentation methods for incorporating azido groups in bacterial capsular polysaccharides, in particular in Streptococcus pneumoniae capsular polysaccharides. The invention also relates to capsular polysaccharides comprising azido groups obtained by said methods. The capsular polysaccharides comprising azido groups may be used for click-based conjugation with alkyne-labeled carrier (e.g. protein carrier) to generate glycoconjugates. The polysaccharides and glycoconjugates obtained by the method may be further used for preparation of immunogenic compositions for preventing and treating infections caused by the organism.

### BACKGROUND OF THE INVENTION

Bacterial cell surface polysaccharides, particularly capsular polysaccharides, have become increasingly important as therapeutic agents. Typically, a cell surface polysaccharide is associated with inducing an immune response in vivo.

Although polysaccharides are immunogenic on their own, conjugation of polysaccharides to protein carriers (glycoconjugate) has been used to improve immunogenicity, particularly in infants and the elderly. Glycoconjugate vaccines are typically obtained by covalent linkage of poorly immunogenic sugar antigens to a protein carrier and play an important role in the prevention of many deadly infectious diseases. In the preparation of conjugate vaccines, selected bacterial strains are grown to supply polysaccharides needed to produce the vaccine. The cells are often grown in fermentors with lysis induced at the end of the fermentation. The lysate broth is then harvested for downstream purification and recovery of the capsular polysaccharide. After conjugation with a carrier protein, the polysaccharide is included in the final vaccine product and confers immunity in the vaccine's target population to the bacteria.

Click conjugation (i.e. conjugation using click chemistry) offers several advantages over the current chemistries used to generate glycoconjugate, such as: increased speed, increased specificity, and lack of organic solvents. However, the azide moiety and alkyne groups which are often used in the click chemistry are absent in almost all naturally existing compounds. Therefore, functionalization of the polysaccharide and the protein carrier are required before conjugation can take place. Introduction of these groups in either the polysaccharide or the carrier protein can be laborious and can introduce structural modification of the polysaccharide which may have an impact on its immunogenicity.

Accordingly, improved methods for functionalization of capsular polysaccharide are needed. In particular methods with reduced number of steps prior to conjugation and which allow for minimal structural modifications of the antigen (i.e. the polysaccharide) are needed.

### SUMMARY OF THE INVENTION

The present invention provides a process for producing a bacterial capsular polysaccharide comprising azido groups, comprising the step of cultivating a capsular polysaccharide-producing bacteria by fermentation in a cell culture media supplemented with a saccharide bearing an azido group.

In an aspect, the process comprises the step of:
(i) inoculating a first container containing a cell culture medium with seed stock of the bacteria, and incubating the first container until growth requirements are met, and
(ii) inoculating a second container containing a cell culture media supplemented with a saccharide bearing an azido group with the culture from step (i), and incubating said second container until growth requirements are met.

In an aspect, the process further comprises the step of purifying the bacterial capsular polysaccharide comprising azido groups.

In an aspect, the cell culture media supplemented with a saccharide bearing an azido group further comprises a growth saccharide.

The invention further relates to an isolated capsular polysaccharide comprising azido groups produced according to said process.

In an aspect, the invention relates to a method of making a glycoconjugate comprising the step of reacting the isolated capsular polysaccharide comprising azido groups with an alkyne functionalized carrier protein by Cu+1 mediated azide-alkyne cycloaddition reaction to form a glycoconjugate.

The invention further provides a glycoconjugate produced according to said method and an immunogenic composition comprising said glycoconjugate.

### FIGURES

FIGS. 1A-1D. Screen for azido sugar incorporation into S. pneumoniae T14 capsule polysaccharide. Vessels (100 mL working volume) were inoculated with a 10-fold dilution of bacteria grown in seed expansion media supplemented with Galactose (30 g/L) (OD600=1.0-1.2) and incubated at 36° C. and 173 rpm with an air overlay of 1.5 SLPH. Media (production medium supplemented with Glucose (30 g/L) (black bars) or production medium supplemented with Galactose (30 g/L) (grey bars)) pH was controlled at 7.0. Bacteria were grown until the ΔOD600≥0 for >30 minutes, then lysed with 0.1% NLS for 30 minutes. Growth was measured offline using a spectrophotometer blanked with PBS at 600 nm. (FIG. 1A) azido incorporation into polysaccharide was then quantified via fluorescent labeling (FL) with normalization to refractive index (RI). MaxOD and End Of Fermentation time (EOF) are provided in (FIG. 1B) and (FIG. 1C), respectively. Polysaccharide was extracted using EtOH and CaCl2 and analyzed via SEC-RI for (FIG. 1D) polysaccharide titer. n=1-4, error bars=+/−StDev.

FIGS. 2A-2D Impact of 2AzGlcAc4 concentration on S. pneumoniae T14 growth and poly production in production medium supplemented with Galactose (30 g/L). Vessels (100 mL working volume) were inoculated with a 10-fold dilution of bacteria grown in seed expansion media supplemented with Galactose (30 g/L) (OD600 nm=1.0-1.2) and incubated at 36° C. and 173 rpm with an air overlay of 1.5 SLPH. Media (production medium supplemented with Galactose (30 g/L)) pH was controlled at 7.0. (FIG. 2A) Growth was measured offline using a spectrophotometer blanked with PBS at 600 nm. At the end of fermentation, cultures were lysed with 0.1% NLS for 30 min. and polysaccharide was extracted via EtOH/CaCl2) precipitation. Polysaccharide titer (FIG. 2B) and size (FIG. 2C) were measured via SEC-RI and SEC-MALS, respectively. (FIG. 2D) azido incorporation was then quantified via fluorescent labeling (FL) and normalized to the refractive index (RI). n=3, error bars=+/−StDev.

FIGS. 3A-3E Impact of 2AzGlc concentration on S. pneumoniae T14 growth and polysaccharide production in production medium supplemented with Galactose (30 g/L). Vessels (100 mL working volume) were inoculated with a 10-fold dilution of bacteria grown in seed expansion media supplemented with Galactose (30 g/L) (OD600 nm=1.0-1.2) and incubated at 36° C. and 173 rpm with an air overlay of 1.5 SLPH. Media (production medium supplemented with Galactose (30 g/L)) pH was controlled at 7.0. (FIG. 3A) Growth was measured offline using a spectrophotometer blanked with PBS at 600 nm. Cultures were lysed with 0.1% NLS for 30 min. and polysaccharide was extracted via EtOH/CaCl2) precipitation. Polysaccharide (FIG. 3B/FIG. 3C) titer and (FIG. 3D) size were measured via SEC-RI and SEC-MALS, respectively. (FIG. 3E) azido incorporation was then quantified via fluorescent labeling (FL) and normalized to the refractive index (RI). n=3, error bars=+/−StDev.

FIGS. 4A-4C Effect of acetylation on azido sugar (5 mM) incorporation into S. pneumoniae T14 polysaccharide. Vessels (100 mL working volume) were inoculated with a 10-fold dilution of bacteria grown in seed expansion media supplemented with Galactose (30 g/L) (OD600 nm=1.0-1.2) and incubated at 36° C. and 173 rpm with an air overlay of 1.5 SLPH. Media (production medium supplemented with Galactose (30 g/L)) pH was controlled at 7.0. All vessels contain 1% DMSO. (FIG. 4A) Growth was measured offline using a spectrophotometer blanked with PBS at 600 nm. Cultures were lysed with 0.1% NLS for 30 min. and polysaccharide was extracted via EtOH/CaCl2) precipitation. (FIG. 4B) Poly titer measured via SEC-RI. (FIG. 4C) Azido incorporation was quantified via fluorescent labeling (FL) and normalized to the refractive index (RI). n=3, error bars=+/−StDev.

FIGS. 5A-5C Effect of acetylation on azido sugar (20 mM) incorporation into S. pneumoniae T14 polysaccharide. Vessels (100 mL working volume) were inoculated with a 10-fold dilution of bacteria grown in seed expansion media supplemented with Galactose (30 g/L) (OD600 nm=1.0-1.2) and incubated at 36° C. and 173 rpm with an air overlay of 1.5 SLPH. Media (production medium supplemented with Galactose (30 g/L)) pH was controlled at 7.0. (FIG. 5A) Growth was measured offline using a spectrophotometer blanked with PBS at 600 nm. Cultures were lysed with 0.1% NLS for 30 min. and polysaccharide was extracted via EtOH/CaCl2) precipitation. (FIG. 5B) Poly titer measured via SEC-RI. (FIG. 5C) Azido incorporation was quantified via fluorescent labeling (FL) and normalized to the refractive index (RI). n=3, error bars=+/−StDev.

FIGS. 6A-6D S. pneumoniae T14 galactose fed-batch fermentation. Fermenters (100 mL working volume) were inoculated with a 10-fold dilution of S. pneumoniae T14 seed bottle culture (seed expansion media supplemented with Galactose, OD600=1.0-1.2) containing production medium media with either 5.0 or 30.0 g/L of galactose. Vessels were incubated at 36° C., at 173 rpm, with an air overlay of 1.5 SLPH. Additional galactose was provided as a feed. (FIG. 6A) Growth was monitored using inline probes and (FIG. 6B) extracellular galactose was measured hourly. Once the ΔOD600 nm≤0 for 1 hr, bacteria were lysed with 0.1% NLS for 30 minutes. Polysaccharide was extracted using EtOH and CaCl2. (FIG. 6C) poly titer and (FIG. 6D) poly size were measured using SEC-RI and SEC-MALS, respectively. n=1.

FIGS. 7A-7C Effect of 2AzGlc on S. pneumoniae T14 grown in a fed-batch. (FIG. 7A) Growth was measured using a spectrophotometer blanked with PBS at 600 nm. Polysaccharide was extracted via EtOH/CaCl2) precipitation. (FIG. 7B) Polysaccharide titer measured via SEC-RI. (FIG. 7C) Azido incorporation was quantified via fluorescent labeling (FL) and normalized to the refractive index (RI). n=1-2, error bars=+/−StDev.

FIGS. 8A-8C Impact of 2AzGlc Titration on S. pneumoniae T14 growth and incorporation in production medium supplemented with glucose (dextrose, 30 g/L). Growth was measured offline using a spectrophotometer blanked with PBS at 600 nm. (FIG. 8A) Media pH=7.0, n=1 for each condition. Media pH=7.0, n=1 for each condition. (FIG. 8B) Media pH=7.33, n=1-2. (FIG. 8C) Incorporation of 2AzGlc into T14 polysaccharide. Non-linear regression fit to sigmoidal curve of combined data sets from panels A and B. Dashed lines represent 95% confidence interval.

### DETAILED DESCRIPTION OF THE INVENTION

Click chemistry often consists in reacting an azido containing moiety with an alkyne containing moiety by Cu+1 mediated azide-alkyne cycloaddition reaction. The click chemistry can be used to obtain glycoconjugates. However, both azido and alkyne groups are absent in almost all naturally existing compounds. Therefore, using click chemistry requires functionalization of the polysaccharide and the protein carrier before conjugation can take place.

Processes used for functionalization, and in particular to functionalize the polysaccharide may be very long tedious processes and may lead to structural modifications of the antigen which may impact the immunogenicity.

The present invention relates to a process for incorporating azido groups in bacterial capsular polysaccharides, in particular in Streptococcus pneumoniae capsular polysaccharides. It has been surprisingly found that supplementing the cell culture medium with sugars comprising an azido group allows for effective incorporation of the azido group into capsule polysaccharide. Hence, the azido labeled polysaccharide can be directly purified from the bacterial cells and subsequently used to generate conjugate by click conjugation.

### 1. Process for Incorporating Azido Groups in Bacterial Capsular Polysaccharides of the Invention

In an embodiment, the present invention is directed to a process for producing a bacterial capsular polysaccharide comprising azido groups, comprising the step of cultivating a capsular polysaccharide-producing bacteria by fermentation in a cell culture media supplemented with a saccharide bearing an azido group.

Before the bacteria are grown in the cell culture media supplemented with a saccharide bearing an azido group, the population of the organism may be scaled up from a seed vial to seed bottles and passaged through one or more seed fermentors of increasing volume until production scale fermentation volumes are reached.

Therefore, in an embodiment, the present invention is directed to a process for producing a bacterial capsular polysaccharide comprising azido groups comprising the steps of cultivating a capsular polysaccharide-producing bacteria in a cell culture medium, which includes: (i) inoculating a first container containing a cell culture medium with seed stock of the bacteria, and incubating the first container until growth requirements are met, and (ii) inoculating a second container containing a cell culture media supplemented with a saccharide bearing an azido group with the culture from step (i), and incubating said second container until growth requirements are met.

In one embodiment, following the culture in the cell culture media supplemented with a saccharide bearing an azido group, the process for producing a bacterial capsular polysaccharide comprising azido groups of the present invention further comprises the step of purifying the bacterial capsular polysaccharide comprising azido groups.

Therefore, in an embodiment, the present invention is directed to a process for producing a bacterial capsular polysaccharide comprising azido groups comprising the steps of:
(a) cultivating a capsular polysaccharide-producing bacteria in a cell culture medium, which includes: (i) inoculating a first container containing a cell culture medium with seed stock of the bacteria, and incubating the first container until growth requirements are met, (ii) inoculating a second container containing a cell culture media supplemented with a saccharide bearing an azido group with the culture from step (i), and incubating said second container until growth requirements are met,
(b) purifying the bacterial capsular polysaccharide from said bacteria, thereby obtaining a bacterial capsular polysaccharide comprising azido groups.

The bacterial culture is typically obtained by batch culture, fed batch culture or continuous culture (see e.g. WO 2007/052168 or WO 2009/081276). Therefore, in an embodiment, the culture in said cell culture media supplemented with a saccharide bearing an azido group is batch culture, fed batch culture or continuous culture. In a preferred embodiment, said culture is batch culture or fed batch culture. In an embodiment, said culture is fed batch culture. In a most preferred embodiment, said culture is batch culture.

In an embodiment, the culture in said cell culture media supplemented with a saccharide bearing an azido group last for between about 2 and about 120 hours. Preferably, the culture in said cell culture media supplemented with a saccharide bearing an azido group last for between about 2 and about 72 hours. Preferably, the culture in said cell culture media supplemented with a saccharide bearing an azido group last for between about 4 and about 12 hours. Even more preferably, the culture in said cell culture media supplemented with a saccharide bearing an azido group last for between about 6 and about 9 hours.

In an embodiment, the culture in said cell culture media supplemented with a saccharide bearing an azido group last until the culture reaches an OD₆₀₀ of between about 4 and about 100. In an embodiment, the culture in said cell culture media supplemented with a saccharide bearing an azido group last until the culture reaches an OD₆₀₀ of between about 4 and about 50. In an embodiment, the culture in said cell culture media supplemented with a saccharide bearing an azido group last until the culture reaches an OD₆₀₀ of between about 4 and about 25. Preferably, the culture in said cell culture media supplemented with a saccharide bearing an azido group last until the culture reaches an OD₆₀₀ of between about 4 and about 16. More preferably, the culture in said cell culture media supplemented with a saccharide bearing an azido group last until the culture reaches an OD₆₀₀ of between about 6 and about 14. Even more preferably, the culture in said cell culture media supplemented with a saccharide bearing an azido group last until the culture reaches an OD₆₀₀ of between about 8 and about 12.

In an embodiment, the culture in said cell culture media supplemented with a saccharide bearing an azido group last until the ΔOD600≤0 over a period of at least 30 minutes.

In an embodiment, the cell culture media supplemented with a saccharide bearing an azido group comprises between 5 mM and 60 mM of saccharide bearing an azido group. In an embodiment, the cell culture media supplemented with a saccharide bearing an azido group comprises between 10 mM and 50 mM of saccharide bearing an azido group. In an embodiment, the cell culture media supplemented with a saccharide bearing an azido group comprises between 15 mM and 45 mM of saccharide bearing an azido group. Preferably, the cell culture media supplemented with a saccharide bearing an azido group comprises between 20 mM and 40 mM of saccharide bearing an azido group. In said embodiments, the culture is preferably a batch culture.

In one embodiment, the cell culture media supplemented with a saccharide bearing an azido group further comprises a growth saccharide. A “growth saccharide” according to the present invention refers to a saccharide present in the medium for the purpose of supporting vigorous growth of the bacteria. The growth saccharide is generally present in abundance at the onset of the fermentation.

In an embodiment, the growth saccharide of the present invention used to supplement the cell culture media is a monosaccharide, an oligosaccharide or a polysaccharide. Preferably, the growth saccharide of the present invention used to supplement the cell culture media is a monosaccharide or an oligosaccharide. In an embodiment, the growth saccharide of the present invention used to supplement the cell culture media is a monosaccharide, a di-saccharide, a tri-saccharide or an oligosaccharide. Preferably, said growth saccharide is a monosaccharide or a di-saccharide. Most preferably, said growth saccharide is a monosaccharide.

In an embodiment, the growth saccharide of the present invention is a trisaccharide. Preferably, said trisaccharide is nigerotriose, maltotriose, melezitose, maltotriulose, raffinose or kestose. Preferably, said trisaccharide is raffinose.

In an embodiment, the growth saccharide of the present invention is a disaccharide. Preferably, said disaccharide is sucrose, maltose, lactose, lactulose or trehalose. Preferably, said disaccharide is sucrose.

In a preferred embodiment, the growth saccharide of the present invention is a monosaccharide. Preferably, said monosaccharide is a triose, a tetrose, a pentose, an hexose or an heptose. More preferably, said monosaccharide is a pentose or an hexose. Most preferably, said monosaccharide is an hexose.

In an embodiment said monosaccharide is a ketose. However, preferably, said monosaccharide is an aldose.

In a preferred embodiment of the present invention, the growth saccharide is an aldopentose or an aldohexose.

In an embodiment of the present invention, the growth saccharide of the present invention is a pentose such as arabinose, lyxose, ribose or xylose. Preferably said saccharide is arabinose or ribose. More preferably though, the growth saccharide of the present invention is an hexose such as glucose, galactose mannose or fructose. Most preferably, the growth saccharide the present invention is glucose or galactose.

In an embodiment of the present invention, the growth saccharide of the present invention is galactose.

In a very preferred embodiment of the present invention, the growth saccharide of the present invention is glucose.

In an embodiment, the growth saccharide of the present invention used to supplement the cell culture media is a racemate. In an embodiment, the growth saccharide of the present invention is a L-isomer. Most preferably, the growth saccharide of the present invention is a D-isomer.

Therefore, in a very preferred embodiment of the present invention, the growth saccharide of the present invention is dextrose.

In an embodiment, the growth saccharide is similar to the saccharide bearing an azido group used to supplement the cell culture media. For example, in case the saccharide bearing an azido group of the present invention is derived from dextrose, then the growth saccharide may be glucose, preferably dextrose.

In an embodiment, said growth saccharide is present at an initial concentration of between about 1.5 g/L and about 60 g/L. In an embodiment, said growth saccharide is present at an initial concentration of between about 10 g/L and about 50 g/L. Preferably, said growth saccharide is present at an initial concentration of between about 20 g/L and about 50 g/L. More Preferably, said growth saccharide is present at an initial concentration of between about 25 g/L and about 50 g/L. Most preferably, said growth saccharide is present at an initial concentration of between about 25 g/L and about 35 g/L.

In an embodiment, said growth saccharide is present at an initial concentration of about 30 g/L.

In an embodiment, the cell culture media supplemented with a saccharide bearing an azido group comprises dextrose as a growth saccharide at an initial concentration of between about 20 g/L and about 50 g/L. More Preferably, at an initial concentration of between about 25 g/L and about 40 g/L. Most preferably, at an initial concentration of between about 25 g/L and about 35 g/L.

In an embodiment, the cell culture media supplemented with a saccharide bearing an azido group comprises galactose as a growth saccharide at an initial concentration of between about 20 g/L and about 50 g/L. More Preferably, at an initial concentration of between about 25 g/L and about 40 g/L. Most preferably, at an initial concentration of between about 25 g/L and about 35 g/L.

In one embodiment, the cell culture media supplemented with a saccharide bearing an azido group is maintained at a pH of between 6.0 and 8.0. Preferably, the pH is maintained between 6.2 and 7.5. More preferably, the pH is maintained between 7.0 and 7.5. Most preferably, the pH is maintained at about 7.33.

In an embodiment, the culture is fed batch and the media supplemented with a saccharide bearing an azido group comprises an initial concentration of saccharide bearing an azido group of between 0.05 mM and 2.5 mM and a feed rate of growth saccharide of between 0.0001 to 0.5 mmol/hr. In an embodiment, the culture is fed batch and the media supplemented with a saccharide bearing an azido group comprises an initial concentration of saccharide bearing an azido group of between 0.05 mM and 2.5 mM and a feed rate of growth saccharide of between 0.001 to 0.05 mmol/hr. Preferably, the culture is fed batch and the media supplemented with a saccharide bearing an azido group comprises an initial concentration of saccharide bearing an azido group of between 0.05 mM and 0.5 mM and a feed rate of growth saccharide of between 0.001 to 0.01 mmol/hr.

Following the culture step which allows for incorporation of azido groups into the capsular polysaccharide, the bacterial capsular polysaccharide comprising azido groups can be purified by a variety of techniques known to the skilled person (see, for example, US2006/0228380, US2006/0228381, WO2008/118752 and WO2020/170190). Therefore, in one embodiment, following the culture in the cell culture media supplemented with a saccharide bearing an azido group, the process for producing a bacterial capsular polysaccharide comprising azido groups of the present invention further comprises the step of purifying the bacterial capsular polysaccharide comprising azido groups.

Generally, a small amount of polysaccharide is released into the cell culture medium during bacterial growth, and so the starting material for purification of the polysaccharide comprising azido groups may be the supernatant from a centrifuged bacterial culture.

Typically, however, the starting material will be prepared by treating the bacteria themselves, such that the polysaccharide comprising azido groups is released.

Optionally, after cell growth, the bacterial cells are deactivated. A suitable method for deactivation is for example treatment with phenol:ethanol, e.g. as described in Fattom et al. (1990) Infect Immun. 58(7):2367-74. In the below embodiments, the bacterial cells may be previously deactivated or not deactivated.

Polysaccharides can be released from bacteria by various methods, including chemical, physical or enzymatic treatment (see e.g.; WO2010151544, WO 2011/051917 or WO2007084856).

In an embodiment, the bacterial cells (deactivated or not deactivated) are treated in suspension in their original culture medium. The purification may therefore start with the cells in suspension in their original culture medium.

In another embodiment the bacterial cells are centrifuged prior to release of capsular polysaccharide comprising azido groups. The purification may therefore start with the cells in the form of a wet cell paste. Alternatively, the cells are treated in a dried form. Typically, however, after centrifugation the bacterial cells are resuspended in an aqueous medium that is suitable for the next step in the process, e.g. in a buffer or in distilled water. The cells may be washed with this medium prior to re-suspension.

In an embodiment, the bacterial cells (e.g. in suspension in their original culture medium, in the form of a wet cell paste, in a dried form or resuspended in an aqueous medium after centrifugation) are treated with a lytic agent. In an embodiment, the bacterial cells in suspension in their original culture medium are treated with a lytic agent. In an embodiment, the bacterial cells resuspended in an aqueous medium after centrifugation are treated with a lytic agent. A “lytic agent” is any agent that aids in cell wall breakdown.

In an embodiment, the lytic agent is a detergent. As used herein, the term “detergent” refers to any anionic or cationic detergent capable of inducing lysis of bacterial cells. Representative examples of such detergents for use within the methods of the present invention include deoxycholate sodium (DOC), N-lauroyl sarcosine (NLS), chenodeoxycholic acid sodium, and saponins (see WO 2008/118752 pages 13 lines 14 to page 14 line 10). In one embodiment of the present invention, the lytic agent used for lysing bacterial cells is DOC. In one embodiment of the present invention, the lytic agent used for lysing bacterial cells is NLS.

In an embodiment, the lytic agent is a non-animal derived lytic agent. In one embodiment, the non-animal derived lytic agent is selected from the group consisting of decanesulfonic acid, tert-octylphenoxy 5 poly(oxyethylene)ethanols (e.g. Igepal® CA-630, CAS #: 9002-93-1, available from Sigma Aldrich, St. Louis, MO), octylphenol ethylene oxide condensates (e.g. Triton® X-100, available from Sigma Aldrich, St. Louis, MO), N-lauroyl sarcosine sodium (NLS), lauryl iminodipropionate, sodium dodecyl sulfate, chenodeoxycholate, hyodeoxycholate, glycodeoxycholate, taurodeoxycholate, taurochenodeoxycholate, and cholate. In one embodiment, the non-animal derived lytic agent is decanesulfonic acid, tert-octylphenoxy 5 poly(oxyethylene)ethanols (e.g. Igepal® CA-630, CAS #: 9002-93-1, available from Sigma Aldrich, St. Louis, MO), octylphenol ethylene oxide condensates (e.g. Triton® X-100, available from Sigma Aldrich, St. Louis, MO), N-lauroyl sarcosine sodium (NLS), lauryl iminodipropionate, sodium dodecyl sulfate, chenodeoxycholate, hyodeoxycholate, glycodeoxycholate, taurodeoxycholate, taurochenodeoxycholate or cholate. In an embodiment, the non-animal derived lytic agent is NLS.

In an embodiment, the bacterial cells (e.g. in suspension in their original culture medium, in the form of a wet cell paste, in a dried form or resuspended in an aqueous medium after centrifugation) are enzymatically treated such that the polysaccharide is released. In an embodiment, the bacterial cells are treated by an enzyme selected from the group consisting of lysostaphin, mutanolysin β-N-acetylglucosaminidase and a combination of mutanolysin and β-N-acetylglucosaminidase. In an embodiment, the bacterial cells are treated by lysostaphin, mutanolysin β-N-acetylglucosaminidase or a combination of mutanolysin and β-N-acetylglucosaminidase. These act on the bacterial peptidoglycan to release the capsular saccharide for use with the invention but also lead to release of the group-specific carbohydrate antigen. In an embodiment, the bacterial cells are treated by a type II phosphodiesterase (PDE2). Optionally, after polysaccharide release, the enzyme(s) is/are deactivated. A suitable method for deactivation is for example heat treatment or acidic treatment.

In an embodiment, the bacterial cells (e.g. in suspension in their original culture medium, in the form of a wet cell paste, in a dried form or resuspended in an aqueous medium after centrifugation) are heated such that the polysaccharide comprising azido groups is released.

In a further embodiment, the bacterial cells (e.g. in suspension in their original culture medium or resuspended in an aqueous medium after centrifugation) are chemically treated such that the polysaccharide comprising azido groups is released. In such an embodiment, the chemical treatment can be for example hydrolysis using base or acid (see e.g. WO2007084856).

Once released, the polysaccharide comprising azido groups may then be isolated from the cell lysate using purification techniques known in the art, including the use of centrifugation, depth filtration, precipitation, ultra-filtration, treatment with activate carbon, diafiltration and/or column chromatography (see, for example, US2006/0228380, US2006/0228381, WO2008/118752 and WO2020/170190). The purified capsular polysaccharide comprising azido groups can then be used for the preparation of immunogenic conjugates.

### 2. Capsular Polysaccharide-Producing Bacteria of the Invention

In an embodiment, the capsular polysaccharide-producing bacteria of the present invention is a pathogenic bacteria. Preferably, the capsular polysaccharide-producing bacteria of the present invention is a pathogenic Streptococcus, a pathogenic Staphylococcus, a pathogenic Enterococcus, a pathogenic Bacillus, a pathogenic Corynebacterium, a pathogenic Listeria, a pathogenic Erysipelothrix, a pathogenic Clostridium, a pathogenic Haemophilus, a pathogenic Neisseria or a pathogenic Escherichia. More preferably, the capsular polysaccharide-producing bacteria of the present invention is a pathogenic Streptococcus, a pathogenic Staphylococcus or a pathogenic Enterococcus.

In an embodiment, the capsular polysaccharide-producing bacteria of the present invention is Aeromonas hydrophila and other species (spp.); Bacillus anthracis; Bacillus cereus; Botulinum neurotoxin producing species of Clostridium; Brucella abortus; Brucella melitensis; Brucella suis; Burkholderia mallei (formally Pseudomonas mallei); Burkholderia pseudomallei (formerly Pseudomonas pseudomallei); Campylobacter jejuni; Chlamydia psittaci; Chlamydia trachomatis, Clostridium botulinum; Clostridium dificile; Clostridium perfringens; Coccidioides immitis; Coccidioides posadasii; Cowdria ruminantium (Heartwater); Coxiella burnetii; Enterococcus faecalis; Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), and Escherichia coli—enteroinvasive (EIEC); Ehrlichia spp. such as Ehrlichia chajfeensis; Francisella tularensis; Legionella pneumophilia; Liberobacter africanus; Liberobacter asiaticus; Listeria monocytogenes; miscellaneous enterics such as Klebsiella, Enterobacter, Proteus, Citrobacter, Aerobacter, Providencia, and Serratia; Mycobacterium bovis; Mycobacterium tuberculosis; Mycoplasma capricolum; Mycoplasma mycoides ssp mycoides; Peronosclerospora philippinensis; Phakopsora pachyrhizi; Plesiomonas shigelloides; Ralstonia solanacearum race 3, biovar 2; Rickettsia prowazekii; Rickettsia rickettsii; Salmonella spp.; Schlerophthora rayssiae var zeae; Shigella spp.; Staphylococcus aureus; Streptococcus; Synchytrium endobioticum; Vibrio cholerae non-01; Vibrio cholerae 01; Vibrio parahaemolyticus and other Vibrios; Vibrio vulnificus; Xanthomonas oryzae; Xylella fastidiosa (citrus variegated chlorosis strain); Yersinia enterocolitica and Yersinia pseudotuberculosis; or Yersinia pestis.

In a preferred embodiment, the capsular polysaccharide-producing bacteria of the present invention is Streptococcus pneumoniae, Streptococcus agalactiae, Streptococcus pyogenes, Staphylococcus aureus, Neisseria meningitidis, Escherichia coli, Salmonella typhi, Haemophilus influenzae, Klebsiella pneumoniae, Enterococcus faecium or Enterococcus faecalis. In an even preferred embodiment, the capsular polysaccharide-producing bacteria of the present invention is Streptococcus pneumoniae, Streptococcus agalactiae, or Streptococcus pyogenes. In a most preferred embodiment, the capsular polysaccharide-producing bacteria of the present invention is Streptococcus pneumoniae.

In an embodiment, the capsular polysaccharide-producing bacteria of the present invention is Staphylococcus aureus. In an embodiment, the capsular polysaccharide-producing bacteria of the present invention is Staphylococcus aureus type 5 or Staphylococcus aureus type 8.

In an embodiment, the capsular polysaccharide-producing bacteria of the present invention is Enterococcus faecalis. In yet a further embodiment, the capsular polysaccharide-producing bacteria of the present invention is Haemophilus influenzae type b.

In a further embodiment, the capsular polysaccharide-producing bacteria of the present invention is Neisseria meningitidis. In an embodiment the capsular polysaccharide-producing bacteria of the present invention is N. meningitidis serogroup A (MenA), N. meningitidis serogroup W135 (MenW135), N. meningitidis serogroup Y (MenY), N. meningitidis serogroup X (MenX) or N. meningitidis serogroup C (MenC). In an embodiment the capsular polysaccharide-producing bacteria of the present invention is N. meningitidis serogroup A (MenA). In an embodiment the capsular polysaccharide-producing bacteria of the present invention is N. meningitidis serogroup W135 (MenW135). In an embodiment the capsular polysaccharide-producing bacteria of the present invention is N. meningitidis serogroup Y (MenY). In an embodiment the capsular polysaccharide-producing bacteria of the present invention is N. meningitidis serogroup C (MenC). In an embodiment the capsular polysaccharide-producing bacteria of the present invention is N. meningitidis serogroup X (MenX).

In a further embodiment, the capsular polysaccharide-producing bacteria of the present invention is Escherichia coli. In a further embodiment, the capsular polysaccharide-producing bacteria of the present invention is Enterococcus faecalis.

In a further embodiment, the capsular polysaccharide-producing bacteria of the present invention is Streptococcus agalactiae (Group B streptococcus (GBS)). In some embodiments, the capsular polysaccharide-producing bacteria of the present invention is Streptococcus agalactiae type Ia, Ib, II, III, IV, V, VI, VII, VIII or IX. In some embodiments, the capsular polysaccharide-producing bacteria of the present invention is Streptococcus agalactiae type Ia, Ib, II, III, IV or V.

In a further embodiment, the capsular polysaccharide-producing bacteria of the present invention is Streptococcus agalactiae (Group B streptococcus (GBS)). In some embodiments, the capsular polysaccharide-producing bacteria of the present invention is Streptococcus agalactiae type Ia, Ib, II, III, IV, V, VI, VII or VIII. In some embodiments, the capsular polysaccharide-producing bacteria of the present invention is Streptococcus agalactiae type Ia, Ib, II, III or V.

In a further embodiment, the capsular polysaccharide-producing bacteria of the present invention is Escherichia coli. In an embodiment, the capsular polysaccharide-producing bacteria of the present invention is an Escherichia coli part of the Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), or Escherichia coli—enteroinvasive (EIEC). In an embodiment, the capsular polysaccharide-producing bacteria of the present invention is an Uropathogenic Escherichia coli (UPEC).

In an embodiment, the capsular polysaccharide-producing bacteria of the present invention is an Escherichia coli serotype selected from the group consisting of serotypes O157:H7, O26:H11, O111:H− and O103:H2. In an embodiment, the capsular polysaccharide-producing bacteria of the present invention is an Escherichia coli serotype selected from the group consisting of serotypes O6:K2:H1 and O18:K1:H7. In an embodiment, the capsular polysaccharide-producing bacteria of the present invention is an Escherichia coli serotype selected from the group consisting of serotypes O45:K1, O17:K52:H18, O19:H34 and O7:K1. In an embodiment, the capsular polysaccharide-producing bacteria of the present invention is an Escherichia coli serotype 0104:H4. In an embodiment, the capsular polysaccharide-producing bacteria of the present invention is an Escherichia coli serotype O1:K12:H7. In an embodiment, the capsular polysaccharide-producing bacteria of the present invention is an Escherichia coli serotype O127:H6. In an embodiment, the capsular polysaccharide-producing bacteria of the present invention is an Escherichia coli serotype O139:H28. In an embodiment, the capsular polysaccharide-producing bacteria of the present invention is an Escherichia coli serotype 0128:H2.

In a preferred embodiment, the capsular polysaccharide-producing bacteria of the present invention is Streptococcus pneumoniae. Preferably, the capsular polysaccharide-producing bacteria of the present invention is Streptococcus pneumoniae serotype 1, 2, 3, 4, 5, 6A, 6B, 6C, 6D, 6E, 6F, 6G, 6H, 7A, 7B, 7C, 7F, 8, 9A, 9L, 9V, 9N, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F or 48.

More preferably, the capsular polysaccharide-producing bacteria of the present invention is Streptococcus pneumoniae serotype 1, 2, 3, 4, 5, 6A, 6B, 6C, 7C, 7F, 8, 9V, 9N, 10A, 10B, 11A, 12F, 14, 15A, 15B, 15C, 16F, 17F, 18C, 19A, 19F, 20A, 20B, 21, 22A, 22F, 23A, 23B, 23F, 24B, 24F, 27, 29, 31, 33B, 33F, 34, 35B, 35F, 38, 45 or 46.

Even more preferably, the capsular polysaccharide-producing bacteria of the present invention is Streptococcus pneumoniae serotype 1, 2, 3, 4, 5, 6A, 6B, 7F, 8, 9V, 9N, 10A, 11A, 12F, 14, 15A, 15B, 15C, 16F, 17F, 18C, 19A, 19F, 20A, 20B, 22F, 23A, 23B, 23F, 24F, 27, 29, 31, 33F or 35B.

Even more preferably, the capsular polysaccharide-producing bacteria of the present invention is Streptococcus pneumoniae serotype 1, 3, 4, 5, 6A, 6B, 7F, 8, 9V, 9N, 10A, 11A, 12F, 14, 15A, 15B, 15C, 18C, 19A, 19F, 22F, 23F, or 33F.

Most preferably, the capsular polysaccharide-producing bacteria of the present invention is Streptococcus pneumoniae serotype 1, 4, 5, 6A, 6B, 7F, 8, 9V, 9N, 10A, 11A, 12F, 14, 15A, 15B, 15C, 18C, 19A, 19F, 22F, 23F, or 33F.

### 3. Saccharide Bearing an Azido Group of the Invention (Used to Supplement the Cell Culture)

In an embodiment, the saccharide bearing an azido group of the present invention used to supplement the cell culture media is a monosaccharide bearing an azido group, a di-saccharide bearing an azido group or a tri-saccharide bearing an azido group. Preferably, said saccharide bearing an azido group is a monosaccharide bearing an azido group or a di-saccharide bearing an azido group. Most preferably, said saccharide bearing an azido group is a monosaccharide bearing an azido group.

In an embodiment, the saccharide bearing an azido group of the present invention is a trisaccharide bearing an azido group. Preferably, said trisaccharide bearing an azido group is derived from nigerotriose, derived from maltotriose, derived from melezitose, derived from maltotriulose, derived from raffinose or derived from kestose. Preferably, said trisaccharide bearing an azido group is derived from raffinose. Where “derived from” means that an azido group has been introduced in said sugar (e.g. “derived from raffinose” is meant to refer to raffinose where an azido group has been introduced).

In an embodiment, the saccharide bearing an azido group of the present invention is a disaccharide bearing an azido group. Preferably, said disaccharide bearing an azido group is derived from sucrose, derived from maltose, derived from lactose, derived from lactulose or derived from trehalose. Preferably, said disaccharide bearing an azido group is derived from sucrose. Where “derived from” means that an azido group has been introduced in said sugar (e.g. derived from sucrose is meant to refer to sucrose where an azido group has been introduced).

In an embodiment, the saccharide bearing an azido group of the present invention is an amino monosaccharide bearing an azido group. Preferably, said amino monosaccharide is an amino hexose bearing an azido group. In an embodiment, the amino hexose is derived from galactosamine or glucosamine. Where “derived from” means that an azido group has been introduced in said amino hexose (it is meant to refer to said amino hexose where an azido group has been introduced). Preferably, the amino hexose is derived from glucosamine. In an embodiment, the saccharide bearing an azido group of the present invention is an N-acetylated monosaccharide bearing an azido group. Preferably, said N-acetylated monosaccharide is a N-acetylated hexose bearing an azido group. In an embodiment, the saccharide bearing an azido group of the present invention is derived from N-acetylgalactosamine, N-acetylglucosamine or N-acetylmannosamine. Where “derived from” means that an azido group has been introduced in said N-acetyl sugar (it is meant to refer to said N-acetyl sugar where an azido group has been introduced). Preferably, the N-acetylated monosaccharide bearing an azido group is N-Azidoacetyl-galactosamine, N-Azidoacetyl-glucosamine or N-Azidoacetyl-mannosamine. More preferably, the N-acetylated monosaccharide bearing an azido group is N-Azidoacetyl-galactosamine or N-Azidoacetyl-glucosamine. Most preferably, the N-acetylated monosaccharide bearing an azido group is N-Azidoacetyl-glucosamine.

In an embodiment, the saccharide bearing an azido group of the present invention is an uronic acid bearing an azido group. Preferably, said uronic acid is an hexuronic acid bearing an azido group. In an embodiment, the hexuronic acid bearing an azido group is derived from glucuronic acid or galacturonic acid. Where “derived from” means that an azido group has been introduced in said hexuronic acid (it is meant to refer to said hexuronic acid where an azido group has been introduced). In an embodiment, the hexuronic acid bearing an azido group of the present invention is derived from glucuronic acid or galacturonic acid and the azido group is located at position C2, C3 or C4. Even more preferably, the azido group is located at position C2 or C3. Preferably, the hexuronic acid is derived from glucuronic acid.

In a preferred embodiment, the saccharide bearing an azido group of the present invention is a monosaccharide. Preferably, said monosaccharide bearing an azido group is derived from a triose, a tetrose, a pentose, an hexose or an heptose. More preferably, said monosaccharide bearing an azido group is derived from a pentose or an hexose. Most preferably, said monosaccharide bearing an azido group is derived from an hexose.

In an embodiment said monosaccharide is derived from a ketose. However, preferably, said monosaccharide is derived from an aldose.

In a preferred embodiment of the present invention, the saccharide bearing an azido group of the present invention is a derived from an aldopentose or an aldohexose.

In an embodiment of the present invention, the saccharide bearing an azido group of the present invention is derived from a pentose such as arabinose, lyxose, ribose or xylose.

Preferably said saccharide is derived from arabinose or ribose. More preferably though, the saccharide bearing an azido group of the present invention is derived from an hexose such as glucose, galactose, mannose or fructose. Most preferably, the saccharide bearing an azido group of the present invention is derived from glucose or galactose.

In a preferred embodiment of the present invention, the saccharide bearing an azido group of the present invention is derived from galactose. Where “derived from” means that an azido group has been introduced in galactose (it is meant to refer to galactose where an azido group has been introduced).

In a very preferred embodiment of the present invention, the saccharide bearing an azido group of the present invention is derived from glucose. Where “derived from” means that an azido group has been introduced in glucose (it is meant to refer to glucose where an azido group has been introduced).

In an embodiment, the saccharide bearing an azido group of the present invention is derived from an hexose and the azido group is located at position C1, C2, C3, C4, C5 or C6. Preferably though, the azido group is located at position C2, C3, C4 or C5. Preferably though, the azido group is located at position C2, C3 or C4. Even more preferably, the azido group is located at position C2 or C3.

In a preferred embodiment, the saccharide bearing an azido group of the present invention is derived from an hexose and the azido group is located at position C3.

In a most preferred embodiment, the saccharide bearing an azido group of the present invention is derived from an hexose and the azido group is located at position C2.

In an embodiment, the saccharide bearing an azido group of the present invention is derived from galactose and the azido group is located at position C1, C2, C3, C4, C5 or C6. Preferably though, the saccharide bearing an azido group of the present invention is derived from galactose and the azido group is located at position C2 or C3. Even more preferably, the saccharide bearing an azido group of the present invention is derived from galactose and the azido group is located at position C2.

In a preferred embodiment, the saccharide bearing an azido group of the present invention is derived from glucose and the azido group is located at position C1, C2, C3, C4, C5 or C6. Preferably though, the saccharide bearing an azido group of the present invention is derived from glucose and the azido group is located at position C2 or C3. Most preferably, the saccharide bearing an azido group of the present invention is derived from glucose and the azido group is located at position C2.

In an embodiment, the saccharide bearing an azido group of the present invention used to supplement the cell culture media is a racemate. In an embodiment, said saccharide bearing an azido group is a L-isomer. Most preferably, said saccharide bearing an azido group is a D-isomer.

In a very preferred embodiment of the present invention, the saccharide bearing an azido group of the present invention is derived from dextrose. Where “derived from” means that an azido group has been introduced in dextrose (it is meant to refer to glucose where an azido group has been introduced).

In a very preferred embodiment, the saccharide bearing an azido group of the present invention is derived from dextrose and the azido group is located at position C2 or C3. Most preferably, the saccharide bearing an azido group of the present invention is derived from dextrose and the azido group is located at position C2.

### 4. Bacterial Capsular Polysaccharide Comprising Azido Groups of the Invention

The process for producing bacterial capsular polysaccharide comprising azido groups allows to produce said polysaccharide by fermentation. Following the culture, the bacterial capsular polysaccharide comprising azido groups can be purified. The isolated capsular polysaccharide comprising azido groups obtained by purification can be characterized by different parameters including, for example the weight average molecular weight (Mw). The molecular weight of the polysaccharide can be measured by Size Exclusion Chromatography (SEC) combined with Multiangle Laser Light Scattering detector (MALLS).

In an aspect, the invention provides an isolated capsular polysaccharide comprising azido groups produced according to any of the methods disclosed herein.

The isolated capsular polysaccharide comprising azido groups of the invention may be characterized by the number of azido groups as a function of repeat units of the saccharide. In one embodiment, the isolated capsular polysaccharide comprising azido groups of the invention comprises at least one azido groups for every 200 saccharide repeat units of the saccharide. In another embodiment, isolated capsular polysaccharide comprising azido groups of the invention comprises at least one azido groups for every 100 saccharide repeat units of the saccharide. In another embodiment, isolated capsular polysaccharide comprising azido groups of the invention comprises about one to ten azido groups for every 100 saccharide repeat units of the saccharide. In another embodiment, isolated capsular polysaccharide comprising azido groups of the invention comprises about one to five azido groups for every 100 saccharide repeat units of the saccharide.

The use of the process of the present invention allows to generate capsular polysaccharide comprising azido groups with no linker (no additional atom) between the capsular polysaccharide and the azido group. In a preferred embodiment, an hydroxyl group of the polysaccharide is replaced by the azide group. Methods of the prior art to introduce functional groups (such as azido group), can introduce structural modification the polysaccharide with potential impact on immunogenicity.

Therefore, in one embodiment, the isolated capsular polysaccharide comprising azido groups of the invention comprises no linker between the capsular polysaccharide and the azido groups.

In an embodiment the isolated capsular polysaccharide comprising azido groups of the invention is a capsular saccharide from a pathogenic bacteria. Preferably, the isolated capsular polysaccharide comprising azido groups of the invention is a capsular saccharide from a pathogenic Streptococcus, a pathogenic Staphylococcus, a pathogenic Enterococcus, a pathogenic Bacillus, a pathogenic Corynebacterium, a pathogenic Listeria, a pathogenic Erysipelothrix, a pathogenic Clostridium, a pathogenic Haemophilus, a pathogenic Neisseria or a pathogenic Escherichia. More preferably, the capsular saccharide used in the present invention is a capsular saccharide from a pathogenic Streptococcus, a pathogenic Staphylococcus or a pathogenic Enterococcus.

In an embodiment, the isolated capsular polysaccharide comprising azido groups of the invention is a capsular saccharide from Aeromonas hydrophila and other species (spp.); Bacillus anthracis; Bacillus cereus; Botulinum neurotoxin producing species of Clostridium; Brucella abortus; Brucella melitensis; Brucella suis; Burkholderia mallei (formally Pseudomonas mallei); Burkholderia pseudomallei (formerly Pseudomonas pseudomallei); Campylobacter jejuni; Chlamydia psittaci; Chlamydia trachomatis, Clostridium botulinum; Clostridium dificile; Clostridium perfringens; Coccidioides immitis; Coccidioides posadasii; Cowdria ruminantium (Heartwater); Coxiella burnetii; Enterococcus faecalis; Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), and Escherichia coli—enteroinvasive (EIEC); Ehrlichia spp. such as Ehrlichia chajfeensis; Francisella tularensis; Legionella pneumophilia; Liberobacter africanus; Liberobacter asiaticus; Listeria monocytogenes; miscellaneous enterics such as Klebsiella, Enterobacter, Proteus, Citrobacter, Aerobacter, Providencia, and Serratia; Mycobacterium bovis; Mycobacterium tuberculosis; Mycoplasma capricolum; Mycoplasma mycoides ssp mycoides; Peronosclerospora philippinensis; Phakopsora pachyrhizi; Plesiomonas shigelloides; Ralstonia solanacearum race 3, biovar 2; Rickettsia prowazekii; Rickettsia rickettsii; Salmonella spp.; Schlerophthora rayssiae var zeae; Shigella spp.; Staphylococcus aureus; Streptococcus; Synchytrium endobioticum; Vibrio cholerae non-01; Vibrio cholerae 01; Vibrio parahaemolyticus and other Vibrios; Vibrio vulnificus; Xanthomonas oryzae; Xylella fastidiosa (citrus variegated chlorosis strain); Yersinia enterocolitica and Yersinia pseudotuberculosis; or Yersinia pestis.

In a preferred embodiment, the isolated capsular polysaccharide comprising azido groups of the invention is a capsular saccharide from Streptococcus pneumoniae, Streptococcus agalactiae, Streptococcus pyogenes, Staphylococcus aureus, Neisseria meningitidis, Escherichia coli, Salmonella typhi, Haemophilus influenzae, Klebsiella pneumoniae, Enterococcus faecium or Enterococcus faecalis. In an even preferred embodiment, the capsular polysaccharide-producing bacteria of the present invention is Streptococcus pneumoniae, Streptococcus agalactiae, or Streptococcus pyogenes. In a most preferred embodiment, the isolated capsular polysaccharide comprising azido groups of the invention is a capsular saccharide from Streptococcus pneumoniae.

In an embodiment, the isolated capsular polysaccharide comprising azido groups of the invention is a capsular saccharide from Staphylococcus aureus. In an embodiment, the isolated capsular polysaccharide comprising azido groups of the invention is a capsular saccharide from Staphylococcus aureus type 5 or Staphylococcus aureus type 8.

In an embodiment, the isolated capsular polysaccharide comprising azido groups of the invention is a capsular saccharide from Enterococcus faecalis. In yet a further embodiment, the isolated capsular polysaccharide comprising azido groups of the invention is a capsular saccharide from Haemophilus influenzae type b.

In a further embodiment, the isolated capsular polysaccharide comprising azido groups of the invention is a capsular saccharide from Neisseria meningitidis. In an embodiment the isolated capsular polysaccharide comprising azido groups of the invention is a capsular saccharide from N. meningitidis serogroup A (MenA), N. meningitidis serogroup W135 (MenW135), N. meningitidis serogroup Y (MenY), N. meningitidis serogroup X (MenX) or N. meningitidis serogroup C (MenC).

In a further embodiment, the isolated capsular polysaccharide comprising azido groups of the invention is a capsular saccharide from Escherichia coli. In a further embodiment, the isolated capsular polysaccharide comprising azido groups of the invention is a capsular saccharide from Enterococcus faecalis.

In a further embodiment, the isolated capsular polysaccharide comprising azido groups of the invention is a capsular saccharide from Streptococcus agalactiae (Group B streptococcus (GBS)). In some embodiments, the isolated capsular polysaccharide comprising azido groups of the invention is a capsular saccharide from Streptococcus agalactiae type Ia, Ib, II, III, IV, V, VI, VII, VIII or IX. In some embodiments, the isolated capsular polysaccharide comprising azido groups of the invention is a capsular saccharide from Streptococcus agalactiae type Ia, Ib, II, III, IV or V.

In a further embodiment, the isolated capsular polysaccharide comprising azido groups of the invention is a capsular saccharide from Streptococcus agalactiae (Group B streptococcus (GBS)). In some embodiments, the isolated capsular polysaccharide comprising azido groups of the invention is a capsular saccharide from Streptococcus agalactiae type Ia, Ib, II, III, IV, V, VI, VII or VIII. In some embodiments, the isolated capsular polysaccharide comprising azido groups of the invention is a capsular saccharide from Streptococcus agalactiae type Ia, Ib, II, III or V.

In a further embodiment, the isolated capsular polysaccharide comprising azido groups of the invention is a capsular saccharide from Escherichia coli. In an embodiment, the isolated capsular polysaccharide comprising azido groups of the invention is a capsular saccharide from an Escherichia coli part of the Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), or Escherichia coli—enteroinvasive (EIEC). In an embodiment, the isolated capsular polysaccharide comprising azido groups of the invention is a capsular saccharide from an Uropathogenic Escherichia coli (UPEC).

In a preferred embodiment, the isolated capsular polysaccharide comprising azido groups of the invention is a capsular saccharide from Streptococcus pneumoniae. Preferably, the isolated capsular polysaccharide comprising azido groups of the invention is a capsular saccharide from Streptococcus pneumoniae serotype 1, 2, 3, 4, 5, 6A, 6B, 6C, 6D, 6E, 6F, 6G, 6H, 7A, 7B, 7C, 7F, 8, 9A, 9L, 9V, 9N, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F or 48.

More preferably, the isolated capsular polysaccharide comprising azido groups of the invention is a capsular saccharide from Streptococcus pneumoniae serotype 1, 2, 3, 4, 5, 6A, 6B, 6C, 7C, 7F, 8, 9V, 9N, 10A, 10B, 11A, 12F, 14, 15A, 15B, 15C, 16F, 17F, 18C, 19A, 19F, 20, 21, 22A, 22F, 23A, 23B, 23F, 24B, 24F, 27, 29, 31, 33B, 33F, 34, 35B, 35F, 38, 45 or 46.

Even more preferably, the isolated capsular polysaccharide comprising azido groups of the invention is a capsular saccharide from Streptococcus pneumoniae serotype 1, 2, 3, 4, 5, 6A, 6B, 7F, 8, 9V, 9N, 10A, 11A, 12F, 14, 15A, 15B, 15C, 16F, 17F, 18C, 19A, 19F, 20A, 20B, 22F, 23A, 23B, 23F, 24F, 27, 29, 31, 33F or 35B.

Even more preferably, the isolated capsular polysaccharide comprising azido groups of the invention is a capsular saccharide from Streptococcus pneumoniae serotype 1, 3, 4, 5, 6A, 6B, 7F, 8, 9V, 9N, 10A, 11A, 12F, 14, 15A, 15B, 15C, 18C, 19A, 19F, 22F, 23F, or 33F.

Most preferably, the isolated capsular polysaccharide comprising azido groups of the invention is a capsular saccharide from Streptococcus pneumoniae serotype 1, 4, 5, 6A, 6B, 7F, 8, 9V, 9N, 10A, 11A, 12F, 14, 15A, 15B, 15C, 18C, 19A, 19F, 22F, 23F, or 33F.

In a preferred embodiment, the isolated capsular polysaccharide comprising azido groups of the invention (purified before further treatment) has a weight average molecular weight between 50 kDa and 5000 kDa. In a preferred embodiment, the isolated capsular polysaccharide comprising azido groups of the invention has a weight average molecular weight between 500 kDa and 5000 kDa. In another preferred embodiment, the isolated capsular polysaccharide comprising azido groups of the invention has a weight average molecular weight between 1000 kDa and 5000 kDa. Any whole number integer within any of the above ranges is contemplated as an embodiment of the disclosure.

5. Capsular Saccharide Glycoconjugates of the Invention Prepared Using Click Chemistry The glycoconjugates of the present invention are prepared using click chemistry. The invention also relates to a method of making a glycoconjugate, as disclosed herein.

Preferably, in order to generate glycoconjugates with advantageous filterability characteristics, immunogenicity and/or yields, sizing of the isolated capsular polysaccharide comprising azido groups to a target molecular weight range is performed prior to the conjugation to a carrier protein.

Advantageously, the size of the isolated capsular polysaccharide comprising azido groups is reduced while preserving critical features of the structure of the polysaccharide. Mechanical or chemical sizing maybe employed.

In an embodiment, the size of the isolated capsular polysaccharide comprising azido groups is reduced by chemical hydrolysis.

Preferably, the size of the isolated capsular polysaccharide comprising azido groups is reduced by mechanical homogenization. In an embodiment, the size of the isolated capsular polysaccharide comprising azido groups is reduced by high pressure homogenization. High pressure homogenization achieves high shear rates by pumping the process stream through a flow path with sufficiently small dimensions. The shear rate is increased by using a larger applied homogenization pressure, and exposure time can be increased by recirculating the feed stream through the homogenizer. The high-pressure homogenization process can be appropriate for reducing the size of the isolated capsular polysaccharide comprising azido groups while preserving the structural features of the saccharide.

In a preferred embodiment, the isolated capsular polysaccharide comprising azido groups is sized to a weight average molecular weight between 10 kDa and 1000 kDa. In an embodiment, the isolated capsular polysaccharide comprising azido groups is sized to a weight average molecular weight between 50 kDa and 500 kDa. In an embodiment, the isolated capsular polysaccharide comprising azido groups is sized to a weight average molecular weight between 50 kDa and 400 kDa. In an embodiment, the isolated capsular polysaccharide comprising azido groups is sized to a weight average molecular weight between 100 kDa and 750 kDa. In an embodiment, the isolated capsular polysaccharide comprising azido groups is sized to a weight average molecular weight between 100 kDa and 500 kDa. In an embodiment, the isolated capsular polysaccharide comprising azido groups is not sized.

In an embodiment, the invention relates to a method of making a glycoconjugate comprising the step of reacting the isolated capsular polysaccharide comprising azido groups of the invention with an alkyne functionalized carrier protein by Cu+1 mediated azide-alkyne cycloaddition reaction to form a glycoconjugate. In a preferred embodiment, said isolated capsular polysaccharide comprising azido groups is sized as described above.

A component of the glycoconjugate is a carrier protein to which the saccharide is conjugated. The terms “protein carrier” or “carrier protein” or “carrier” may be used interchangeably herein. Carrier proteins should be amenable to standard conjugation procedures.

In a preferred embodiment, the carrier protein of the glycoconjugate of the invention is selected in the group consisting of: DT (Diphtheria toxoid), TT (tetanus toxoid) or fragment C of TT, CRM₁₉₇ (a nontoxic but antigenically identical variant of diphtheria toxin), other DT mutants (such as CRM176, CRM228, CRM45 (Uchida et al. (1973) J. Biol. Chem. 218:3838-3844), CRM9, CRM102, CRM103 or CRM107; and other mutations described by Nicholls and Youle in Genetically Engineered Toxins, Ed: Frankel, Maecel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substution at glutamic acid 553 (Douglas et al. (1987) J. Bacteriol. 169(11):4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa. Another suitable carrier protein is a C5a peptidase from Streptococcus (SCP).

In a preferred embodiment, the carrier protein of the glycoconjugate of the invention is DT, TT, CRM₁₉₇, detoxified pneumococcal pneumolysin, PhtA, PhtB, PhtD, PhtE, OMPC, PorB, PD, PspA, PsaA or C5a peptidase from Streptococcus (SCP). In a more preferred embodiment, the carrier protein of the glycoconjugate of the invention is TT, DT, DT mutants (such as CRM₁₉₇) or a C5a peptidase from Streptococcus (SCP).

In an embodiment, the carrier protein of the glycoconjugate of the invention is DT (Diphtheria toxoid). In another embodiment, the carrier protein of the glycoconjugate of the invention is TT (tetanus toxoid).

In another embodiment, the carrier protein of the glycoconjugate of the invention is PD (H. influenzae protein D; see, e.g., EP0594610 B).

In a preferred embodiment, the carrier protein of the glycoconjugate of the invention is CRM197 or a C5a peptidase from Streptococcus (SCP).

In a most preferred embodiment, the carrier protein of the glycoconjugate of the invention is CRM₁₉₇. The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage β197tox-created by nitrosoguanidine mutagenesis of the toxigenic corynephage beta (Uchida et al. (1971) Nature New Biology 233:8-11). The CRM197 protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382.

In an embodiment, the carrier protein of the glycoconjugate of the invention is the A chain of CRM197 (see CN103495161). In an embodiment, the carrier protein of the glycoconjugate of the invention is the A chain of CRM197 obtained via expression by genetically recombinant E. coli (see CN103495161).

In other preferred embodiments, the carrier protein of the glycoconjugate of the invention is SCP (Streptococcal C5a Peptidase).

In order to be used in the method of making a conjugate of the present invention, the carrier protein may be activated to provide an alkyne functionalized carrier protein. Alkyne functionalized carrier protein refers to a protein carrier bearing at least one terminal alkyne which can react with the azido groups of the isolated capsular polysaccharide comprising azido groups of the invention. Alkyne functionalized carrier proteins are known in the art (see e.g. Crotti S et al., ChemBioChem 2014, 15, 836-843; McKay C and Finn M, Chemistry & Biology, 21, 1075-1101, 2014; Bioconjugate Techniques, Greg T. Hermanson, Third Edition, 2013, Academic Press, Chapter 18 p. 787-838, ISBN 978-0-12-382239-0; and references cited therein).

Therefore, in an embodiment, the carrier protein used in the method of making a conjugate of the present invention is an alkyne functionalized carrier protein. In one embodiment, the alkyne functionalized carrier protein bears a propargyl group.

An alkyne group in the carrier protein is ideal for reacting with an azido group of the isolated capsular polysaccharide comprising azido groups of the invention e.g. using the reactions known in the art as copper-catalyzed azide-alkyne cycloaddition.

In an embodiment, the invention relates to a method of making a glycoconjugate comprising the step of (a) reacting the isolated capsular polysaccharide comprising azido groups of the invention with an alkyne functionalized carrier protein by Cu+1 mediated azide-alkyne cycloaddition reaction to form a glycoconjugate. In a preferred embodiment, said isolated capsular polysaccharide comprising azido groups is sized as described above.

As mentioned above, before the reaction (a), sizing of the capsular polysaccharide to a target molecular weight (MW) range can be performed. Therefore, in an embodiment, the the isolated capsular polysaccharide comprising azido groups is sized before being reacted. In an embodiment, the isolated capsular polysaccharide comprising azido groups is sized to any of the target molecular weight (MW) range defined above.

Following the click conjugation reaction, there may remain unreacted azido groups in the conjugates, these may be capped using a suitable azido group capping agent. Therefore, in an embodiment, following step (a), unreacted azido groups in the conjugate, are capped using a suitable azido group capping agent. In one embodiment this azido group capping agent is an agent bearing an alkyne group. In one embodiment this azido group capping agent is an agent bearing a terminal alkyne. In one embodiment this azido group capping agent is an agent bearing a cycloalkyne. In one embodiment this azido group capping agent is propargyl alcohol.

Following the click conjugation reaction, unreacted alkyne groups may also remain present in the conjugates, these may be capped using a suitable alkyne group capping agent. In one embodiment this alkyne group capping agent is an agent bearing an azido group. In one embodiment this alkyne group capping agent is 3-azido-1-propanol. Therefore, in an embodiment, following step (a) the method further comprises a step of capping the unreacted alkyne groups remained in the conjugates with an alkyne group capping agent.

In an aspect, the invention provides a glycoconjugate produced according to any of the methods disclosed herein.

### 6. Immunogenic Compositions

In an embodiment the invention relates to an immunogenic composition comprising a glycoconjugate of the invention (as disclosed at section 5 above).

In an embodiment the invention relates to an immunogenic composition comprising a glycoconjugate of the invention and comprising from 1 to 25 different glycoconjugates.

Preferably, the invention relates to an immunogenic composition comprising a glycoconjugate of the invention which is a 3 to 25 valent immunogenic composition. In an embodiment, the invention relates to an immunogenic composition comprising a glycoconjugate of the invention which is a 15-valent immunogenic composition. In an embodiment, the invention relates to an immunogenic composition comprising a glycoconjugate of the invention which is a 20-valent immunogenic composition. In an embodiment, the invention relates to an immunogenic composition comprising a glycoconjugate of the invention which is a 21-valent immunogenic composition.

In an embodiment the invention relates to an immunogenic composition comprising a glycoconjugate of the invention and comprising from 1 to 25 glycoconjugates from different serotypes of S. pneumoniae (1 to 25 pneumococcal conjugates). In a preferred embodiment, the invention relates to an immunogenic composition comprising a glycoconjugate of the invention and comprising seven conjugates or more. In one embodiment the invention relates to an immunogenic composition comprising glycoconjugates from 7, 8, 9, 10, 11, 12, 13, 14, 15, 16, 17, 18, 19, 20, 21, 22, 23, 24 or 25 different serotypes of S. pneumoniae. In one embodiment the invention relates to an immunogenic composition comprising glycoconjugates from 15, 16, 17, 18, 19, 20, 21, 22, 23, 24 or 25 different serotypes of S. pneumoniae.

In one embodiment the invention relates to an immunogenic composition comprising glycoconjugates from 15 different serotypes of S. pneumoniae.

In one embodiment the invention relates to an immunogenic composition comprising glycoconjugates from 20 different serotypes of S. pneumoniae.

In one embodiment the invention relates to an immunogenic composition comprising glycoconjugates from 21 different serotypes of S. pneumoniae.

In one embodiment the invention relates to an immunogenic composition comprising glycoconjugates from 22 different serotypes of S. pneumoniae.

In a preferred embodiment, the saccharides are each individually conjugated to different molecules of the protein carrier (each molecule of protein carrier only having one type of saccharide conjugated to it). In said embodiment, the capsular saccharides are said to be individually conjugated to the carrier protein. Preferably, all the glycoconjugates of the above immunogenic compositions are individually conjugated to the carrier protein.

In an embodiment of any of the above immunogenic compositions, the glycoconjugate of the invention is conjugated to CRM₁₉₇. In an embodiment of any of the above immunogenic compositions, the glycoconjugate of the invention is conjugated to SCP.

In an embodiment of any of the above immunogenic compositions, the glycoconjugates of any of the above immunogenic compositions are all individually conjugated to CRM₁₉₇.

In an embodiment of any of the above immunogenic compositions, the glycoconjugate of the invention is conjugated to SCP and the other glycoconjugate(s) is/are all individually conjugated to CRM₁₉₇.

In an embodiment the above immunogenic compositions comprise from 8 to 25 different serotypes of S. pneumoniae.

Compositions of the invention may include a small amount of free carrier. When a given carrier protein is present in both free and conjugated form in a composition of the invention, the unconjugated form is preferably no more than 5% of the total amount of the carrier protein in the composition as a whole, and more preferably present at less than 2% by weight.

The amount of glycoconjugate(s) in each dose is selected as an amount which induces an immunoprotective response without significant, adverse side effects in typical vaccinees. Such amount will vary depending upon which specific immunogen is employed and how it is presented.

The amount of a particular glycoconjugate in an immunogenic composition can be calculated based on total saccharide for that conjugate (conjugated and non-conjugated). For example, a glycoconjugate with 20% free saccharide will have about 80 μg of conjugated saccharide and about 20 μg of nonconjugated saccharide in a 100 μg saccharide dose. The amount of glycoconjugate can vary depending upon the bacteria and bacteria serotype. The saccharide concentration can be determined by the uronic acid assay.

The “immunogenic amount” of the different saccharide components in the immunogenic composition, may diverge and each may comprise about 0.5 μg, about 0.75 μg, about 1 μg, about 2 μg, about 3 μg, about 4 μg, about 5 μg, about 6 μg, about 7 μg, about 8 μg, about 9 μg, about 10 μg, about 15 μg, about 20 μg, about 30 μg, about 40 μg, about 50 μg, about 60 μg, about 70 μg, about 80 μg, about 90 μg, or about 100 μg of any particular saccharide antigen.

Generally, each dose will comprise 0.1 μg to 100 μg of saccharide for a given bacteria or serotype. In an embodiment each dose will comprise 0.1 μg to 100 μg of saccharide for a given bacteria or serotype. In a preferred embodiment each dose will comprise 0.5 μg to 20 μg. In a preferred embodiment each dose will comprise 1.0 μg to 10 μg. In an even preferred embodiment, each dose will comprise 2.0 μg to 5.0 μg of saccharide for a given bacteria or serotype. Any whole number integer within any of the above ranges is contemplated as an embodiment of the disclosure.

Generally, each dose will comprise 10 μg to 150 μg of carrier protein, particularly 15 μg to 100 μg of carrier protein, more particularly 25 μg to 75 μg of carrier protein, and even more particularly 40 μg to 60 μg of carrier protein.

Immunogenic compositions of the invention comprise conjugated saccharide antigen(s) (glycoconjugate(s)). They may also further include antigen(s) from other pathogen(s), particularly from bacteria and/or viruses. Preferred further antigens are selected from: a diphtheria toxoid (D), a tetanus toxoid (T), a pertussis antigen (P), which is typically acellular (Pa), a hepatitis B virus (HBV) surface antigen (HBsAg), a hepatitis A virus (HAV) antigen, a conjugated Haemophilus influenzae type b capsular saccharide (Hib), inactivated poliovirus vaccine (IPV).

In an embodiment, the immunogenic compositions of the invention comprise D-T-Pa. In an embodiment, the immunogenic compositions of the invention comprise D-T-Pa-Hib, D-T-Pa-IPV or D-T-Pa-HBsAg. In an embodiment, the immunogenic compositions of the invention comprise D-T-Pa-HBsAg-IPV or D-T-Pa-HBsAg-Hib. In an embodiment, the immunogenic compositions of the invention comprise D-T-Pa-HBsAg—IPV-Hib.

In some embodiments, the immunogenic compositions disclosed herein may further comprise at least one, two or three adjuvants. In some embodiments, the immunogenic compositions disclosed herein may further comprise at least one adjuvant. In some embodiments, the immunogenic compositions disclosed herein may further comprise one adjuvant. In some embodiments, the immunogenic compositions disclosed herein may further comprise two adjuvants. The term “adjuvant” refers to a compound or mixture that enhances the immune response to an antigen. Antigens may act primarily as a delivery system, primarily as an immune modulator or have strong features of both.

Suitable adjuvants include those suitable for use in mammals, including humans. Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

In an embodiment, the immunogenic compositions disclosed herein comprise aluminum salts (alum) as adjuvant (e.g., aluminum phosphate, aluminum sulfate or aluminum hydroxide). In a preferred embodiment, the immunogenic compositions disclosed herein comprise aluminum phosphate or aluminum hydroxide as adjuvant. In a preferred embodiment, the immunogenic compositions disclosed herein comprise aluminum phosphate as adjuvant.

Further exemplary adjuvants to enhance effectiveness of the immunogenic compositions as disclosed herein include, but are not limited to: (1) oil-in-water emulsion formulations (with or without other specific immunostimulating agents such as muramyl peptides (see below) or bacterial cell wall components), such as for example (a) SAF, containing 10% Squalene, 0.4% Tween 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, MT) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, MA), ABISCO® (Isconova, Sweden), or ISCOMATRIX® (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB-2220221, EP0689454), optionally in the substantial absence of alum when used with pneumococcal saccharides (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+IM2 (optionally+a sterol) (e.g., WO 98/57659); (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc.

In an embodiment of the present invention, the immunogenic compositions as disclosed herein comprise a CpG Oligonucleotide as adjuvant. A CpG oligonucleotide as used herein refers to an immunostimulatory CpG oligodeoxynucleotide (CpG ODN), and accordingly these terms are used interchangeably unless otherwise indicated. Immunostimulatory CpG oligodeoxynucleotides contain one or more immunostimulatory CpG motifs that are unmethylated cytosine-guanine dinucleotides, optionally within certain preferred base contexts. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9). In another embodiment the immunostimulatory oligonucleotide may contain one or more methylated CpG dinucleotides, which will activate the immune system through TLR9 but not as strongly as if the CpG motif(s) was/were unmethylated. CpG immunostimulatory oligonucleotides may comprise one or more palindromes that in turn may encompass the CpG dinucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068.

The immunogenic compositions of the invention may be formulated in liquid form (i.e., solutions or suspensions) or in a lyophilized form. In an embodiment, the immunogenic composition of the invention is formulated in a liquid form. In an embodiment, the immunogenic composition of the invention is formulated in a lyophilized form. Liquid formulations may advantageously be administered directly from their packaged form and are thus ideal for injection without the need for reconstitution in aqueous medium as otherwise required for lyophilized compositions of the invention.

Formulation of the immunogenic composition of the present disclosure can be accomplished using art-recognized methods. For instance, the individual polysaccharides and/or conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

The present disclosure provides an immunogenic composition comprising any of combination of glycoconjugates disclosed herein and a pharmaceutically acceptable excipient, carrier, or diluent.

In an embodiment, the immunogenic composition of the disclosure is in liquid form, preferably in aqueous liquid form.

Immunogenic compositions of the disclosure may comprise one or more of a buffer, a salt, a divalent cation, a non-ionic detergent, a cryoprotectant such as a sugar, and an anti-oxidant such as a free radical scavenger or chelating agent, or any multiple combinations thereof.

In an embodiment, the immunogenic compositions of the disclosure comprise a buffer. In an embodiment, said buffer has a pKa of about 3.5 to about 7.5. In some embodiments, the buffer is phosphate, succinate, histidine or citrate. In some embodiments, the buffer is succinate. In some embodiments, the buffer is histidine. In certain embodiments, the buffer is succinate at a final concentration of 1 mM to 10 mM. In one particular embodiment, the final concentration of the succinate buffer is about 5 mM.

In an embodiment, the immunogenic compositions of the disclosure comprise a salt. In some embodiments, the salt is selected from the groups consisting of magnesium chloride, potassium chloride, sodium chloride and a combination thereof. In one particular embodiment, the salt is sodium chloride. In one particular embodiment, the immunogenic compositions of the invention comprise sodium chloride at 150 mM.

In an embodiment, the immunogenic compositions of the disclosure comprise a surfactant. In an embodiment, the surfactant is selected from the group consisting of polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer.

In one particular embodiment, the surfactant is polysorbate 80. In some said embodiments, the final concentration of polysorbate 80 in the formulation is at least 0.001% to 1% polysorbate 80 weight to weight (w/w). In some said embodiments, the final concentration of polysorbate 80 in the formulation is at least 0.01% to 1% polysorbate 80 weight to weight (w/w).

In one particular embodiment, the surfactant is polysorbate 20. In some said embodiments, the final concentration of polysorbate 20 in the formulation is at least 0.001% to 1% polysorbate 20 weight to weight (w/w). In some said embodiments, the final concentration of polysorbate 20 in the formulation is at least 0.01% to 1% polysorbate 20 weight to weight (w/w).

In one embodiment, the present disclosure provides a container filled with any of the immunogenic compositions disclosed herein. In one embodiment, the container is selected from the group consisting of a vial, a syringe, a flask, a fermentor, a bioreactor, a bag, a jar, an ampoule, a cartridge and a disposable pen. In certain embodiments, the container is siliconized.

In an embodiment, the container of the present disclosure is made of glass, metals (e.g., steel, stainless steel, aluminum, etc.) and/or polymers (e.g., thermoplastics, elastomers, thermoplastic-elastomers). In an embodiment, the container of the present disclosure is made of glass.

In one embodiment, the present disclosure provides a syringe filled with any of the immunogenic compositions disclosed herein. In certain embodiments, the syringe is siliconized and/or is made of glass.

A typical dose of the immunogenic composition of the invention for injection has a volume of 0.1 mL to 2 mL. In an embodiment, the immunogenic composition of the invention for injection has a volume of 0.2 mL to 1 mL, even more preferably a volume of about 0.5 mL.

### 7. Uses of the Glycoconjugate and Immunogenic Compositions of the Invention

The glycoconjugates disclosed herein may be use as antigens. For example, they may be part of a vaccine.

Therefore, in an embodiment, the immunogenic compositions of the invention are for use as a medicament.

In an embodiment, the immunogenic compositions of the invention are for use as a vaccine.

Therefore, in an embodiment, the immunogenic compositions described herein are for use in generating an immune response in a subject. In one aspect, the subject is a mammal, such as a human, non-human primate, cat, sheep, pig, horse, bovine or dog. In one aspect, the subject is a human.

The immunogenic compositions described herein may be used in therapeutic or prophylactic methods for preventing, treating or ameliorating a bacterial infection, disease or condition in a subject. Thus, in one aspect, the disclosure provides a method of preventing, treating or ameliorating an infection, disease or condition associated with a bacterial infection in a subject, comprising administering to the subject an immunologically effective amount of an immunogenic composition of the disclosure.

The immunogenic composition of the present disclosure can be used to protect or treat a human susceptible to a bacterial infection, by means of administering the immunogenic composition via a systemic or mucosal route. In an embodiment, the immunogenic composition of the invention is administered by intramuscular, intraperitoneal, intradermal or subcutaneous routes. In an embodiment, the immunogenic composition of the invention is administered by intramuscular, intraperitoneal, intradermal or subcutaneous injection. In an embodiment, the immunogenic composition of the invention is administered by intramuscular or subcutaneous injection. In an embodiment, the immunogenic composition of the invention is administered by intramuscular injection. In an embodiment, the immunogenic composition of the invention is administered by subcutaneous injection.

As disclosed herein, the immunogenic compositions described herein may be used in various therapeutic or prophylactic methods for preventing, treating or ameliorating a bacterial infection, disease or condition in a subject.

In a preferred embodiment, said subject is a human. In a most preferred embodiment, said subject is a newborn (i.e., under three months of age), an infant (i.e., from 3 months to one year of age) or a toddler (i.e., from one year to four years of age).

In an embodiment, the immunogenic compositions disclosed herein are for use as a vaccine.

In such embodiment, the subject to be vaccinated may be less than 1 year of age.

In an embodiment of the present invention, the subject to be vaccinated is a human adult 50 years of age or older, more preferably a human adult 55 years of age or older. In an embodiment, the subject to be vaccinated is a human adult 65 years of age or older, 70 years of age or older, 75 years of age or older or 80 years of age or older.

In an embodiment the subject to be vaccinated is an immunocompromised individual, in particular a human. An immunocompromised individual is generally defined as a person who exhibits an attenuated or reduced ability to mount a normal humoral or cellular defense to challenge by infectious agents.

### 8. The Invention Also Provides the Following Embodiments as Defined in the Following Numbered Paragraphs 1 to 208

1. A process for producing a bacterial capsular polysaccharide comprising azido groups, comprising the step of cultivating a capsular polysaccharide-producing bacteria by fermentation in a cell culture media supplemented with a saccharide bearing an azido group.
2. The process of paragraph 1 comprising the step of:
   (i) inoculating a first container containing a cell culture medium with seed stock of the bacteria, and incubating the first container until growth requirements are met, and
   (ii) inoculating a second container containing a cell culture media supplemented with a saccharide bearing an azido group with the culture from step (i), and incubating said second container until growth requirements are met.
3. The process of paragraph 1 which further comprises the step of purifying the bacterial capsular polysaccharide comprising azido groups.
4. The process of paragraph 1 comprising the step of:
   (a) cultivating a capsular polysaccharide-producing bacteria in a cell culture medium, which includes: (i) inoculating a first container containing a cell culture medium with seed stock of the bacteria, and incubating the first container until growth requirements are met, (ii) inoculating a second container containing a cell culture media supplemented with a saccharide bearing an azido group with the culture from step (i), and incubating said second container until growth requirements are met,
   (b) purifying the bacterial capsular polysaccharide from said bacteria, thereby obtaining a bacterial capsular polysaccharide comprising azido groups.
5. The process of any one of paragraphs 1-4, wherein the culture in said cell culture media supplemented with a saccharide bearing an azido group is batch culture, fed batch culture or continuous culture.
6. The process of any one of paragraphs 1-4, wherein the culture in said cell culture media supplemented with a saccharide bearing an azido group is batch culture.
7. The process of any one of paragraphs 1-4, wherein the culture in said cell culture media supplemented with a saccharide bearing an azido group is fed batch culture.
8. The process of any one of paragraphs 1-7, wherein the culture in said cell culture media supplemented with a saccharide bearing an azido group last for between about 2 and about 120 hours.
9. The process of any one of paragraphs 1-7, wherein the culture in said cell culture media supplemented with a saccharide bearing an azido group last for between about 2 and about 72 hours
10. The process of any one of paragraphs 1-7, wherein the culture in said cell culture media supplemented with a saccharide bearing an azido group last for between about 4 and about 12 hours
11. The process of any one of paragraphs 1-7, wherein the culture in said cell culture media supplemented with a saccharide bearing an azido group last for between about 6 and about 9 hours
12. The process of any one of paragraphs 1-11, wherein the culture in said cell culture media supplemented with a saccharide bearing an azido group last until the culture reaches an OD₆₀₀ of between about 4 and about 100.
13. The process of any one of paragraphs 1-11, wherein the culture in said cell culture media supplemented with a saccharide bearing an azido group last until the culture reaches an OD₆₀₀ of between about 4 and about 25.
14. The process of any one of paragraphs 1-11, wherein the culture in said cell culture media supplemented with a saccharide bearing an azido group last until the culture reaches an OD₆₀₀ of between about 4 and about 16.
15. The process of any one of paragraphs 1-11, wherein the culture in said cell culture media supplemented with a saccharide bearing an azido group last until the culture reaches an OD₆₀₀ of between about 6 and about 14.
16. The process of any one of paragraphs 1-11, wherein the culture in said cell culture media supplemented with a saccharide bearing an azido group last until the culture reaches an OD₆₀₀ of between about 8 and about 12.
17. The process of any one of paragraphs 1-11, wherein the culture in said cell culture media supplemented with a saccharide bearing an azido group last until the ΔOD600≥0 over a period of at least 30 minutes.
18. The process of any one of paragraphs 1-17, wherein the cell culture media supplemented with a saccharide bearing an azido group comprises between 5 mM and 60 mM of saccharide bearing an azido group.
19. The process of any one of paragraphs 1-17, wherein the cell culture media supplemented with a saccharide bearing an azido group comprises between 10 mM and 50 mM of saccharide bearing an azido group.
20. The process of any one of paragraphs 1-17, wherein the cell culture media supplemented with a saccharide bearing an azido group comprises between 15 mM and 45 mM of saccharide bearing an azido group.
21. The process of any one of paragraphs 1-17, wherein the cell culture media supplemented with a saccharide bearing an azido group comprises between 20 mM and 40 mM of saccharide bearing an azido group.
22. The process of any one of paragraphs 1-21, wherein the cell culture media supplemented with a saccharide bearing an azido group further comprises a growth saccharide
23. The process of paragraph 22, wherein said growth saccharide is a monosaccharide, an oligosaccharide or a polysaccharide.
24. The process of paragraph 22, wherein said growth saccharide is a monosaccharide, a di-saccharide, a tri-saccharide or an oligosaccharide.
25. The process of paragraph 22, wherein said growth saccharide is a monosaccharide or a di-saccharide.
26. The process of paragraph 22, wherein said growth saccharide is a monosaccharide.
27. The process of paragraph 22, wherein said growth saccharide is a monosaccharide or an oligosaccharide.
28. The process of paragraph 22, wherein said growth saccharide is a trisaccharide.
29. The process of paragraph 22, wherein said growth saccharide is nigerotriose, maltotriose, melezitose, maltotriulose, raffinose or kestose.
30. The process of paragraph 22, wherein said growth saccharide is raffinose.
31. The process of paragraph 22, wherein said growth saccharide is a disaccharide.
32. The process of paragraph 22, wherein said growth saccharide is sucrose, maltose, lactose, lactulose or trehalose.
33. The process of paragraph 22, wherein said growth saccharide is sucrose.
34. The process of paragraph 22, wherein said growth saccharide is a triose, a tetrose, a pentose, an hexose or an heptose.
35. The process of paragraph 22, wherein said growth saccharide is a pentose or an hexose.
36. The process of paragraph 22, wherein said growth saccharide is an hexose.
37. The process of paragraph 22, wherein said growth saccharide is a ketose.
38. The process of paragraph 22, wherein said growth saccharide is an aldose.
39. The process of paragraph 22, wherein said growth saccharide is an aldopentose or an aldohexose.
40. The process of paragraph 22, wherein said growth saccharide is a pentose.
41. The process of paragraph 22, wherein said growth saccharide is arabinose, lyxose, ribose or xylose.
42. The process of paragraph 22, wherein said growth saccharide is arabinose or ribose.
43. The process of paragraph 22, wherein said growth saccharide is glucose, galactose or fructose.
44. The process of paragraph 22, wherein said growth saccharide is glucose or galactose.
45. The process of paragraph 22, wherein said growth saccharide is galactose.
46. The process of paragraph 22, wherein said growth saccharide is glucose.
47. The process of any one of paragraphs 22-46, wherein said growth saccharide is a racemate.
48. The process of any one of paragraphs 22-46, wherein said growth saccharide is a L-isomer.
49. The process of any one of paragraphs 22-46, wherein said growth saccharide is a D-isomer.
50. The process of paragraph 22, wherein said growth saccharide is dextrose.
51. The process of any one of paragraphs 22-50, wherein the growth saccharide is similar to the saccharide bearing an azido group used to supplement the cell culture media.
52. The process of any one of paragraphs 22-51, wherein said growth saccharide is present at initial a concentration of between about 1.5 g/L and about 60 g/L.
53. The process of any one of paragraphs 22-51, wherein said growth saccharide is present at initial a concentration of between about 10 g/L and about 50 g/L.
54. The process of any one of paragraphs 22-51, wherein said growth saccharide is present at initial a concentration of between about 20 g/L and about 50 g/L
55. The process of any one of paragraphs 22-51, wherein said growth saccharide is present at initial a concentration of between about 25 g/L and about 50 g/L.
56. The process of any one of paragraphs 22-51, wherein said growth saccharide is present at initial a concentration of between about 25 g/L and about 35 g/L.
57. The process of any one of paragraphs 22-51, wherein said growth saccharide is present at initial a concentration of about 30 g/L.
58. The process of any one of paragraphs 22-51, wherein the cell culture media supplemented with a saccharide bearing an azido group comprises dextrose as a growth saccharide at an initial concentration of between about 20 g/L and about 50 g/L.
59. The process of paragraph 58, wherein the initial concentration of dextrose is between about 25 g/L and about 40 g/L.
60. The process of paragraph 58, wherein the initial concentration of dextrose is between about 25 g/L and about 35 g/L.
61. The process of any one of paragraphs 22-51, wherein the cell culture media supplemented with a saccharide bearing an azido group comprises galactose as a growth saccharide at an initial concentration of between about 20 g/L and about 50 g/L.
62. The process of paragraph 61, wherein the initial concentration of galactose is between about 25 g/L and about 40 g/L.
63. The process of paragraph 61, wherein the initial concentration of galactose is between about 25 g/L and about 35 g/L.
64. The process of any one of paragraphs 1-63, wherein the cell culture media supplemented with a saccharide bearing an azido group is maintained at a pH of between 6.0 and 8.0.
65. The process of any one of paragraphs 1-63, wherein the cell culture media supplemented with a saccharide bearing an azido group is maintained at a pH of between 6.2 and 7.5.
66. The process of any one of paragraphs 1-63, wherein the cell culture media supplemented with a saccharide bearing an azido group is maintained at a pH of between 7.0 and 7.5.
67. The process of any one of paragraphs 1-63, wherein the cell culture media supplemented with a saccharide bearing an azido group is maintained at a pH of about 7.33.
68. The process of any one of paragraphs 1-5, wherein the culture is fed batch and the media supplemented with a saccharide bearing an azido group comprises an initial concentration of saccharide bearing an azido group of between 0.05 mM and 2.5 mM and a feed rate of growth saccharide of between 0.0001 to 0.5 mmol/hr.
69. The process of any one of paragraphs 1-5, wherein the culture is fed batch and the media supplemented with a saccharide bearing an azido group comprises an initial concentration of saccharide bearing an azido group of between 0.05 mM and 2.5 mM and a feed rate of growth saccharide of between 0.001 to 0.05 mmol/hr.
70. The process of any one of paragraphs 1-5, wherein the culture is fed batch and the media supplemented with a saccharide bearing an azido group comprises an initial concentration of saccharide bearing an azido group of between 0.05 mM and 0.5 mM and a feed rate of growth saccharide of between 0.001 to 0.01 mmol/hr.
71. The process of any one of paragraphs 1-70, wherein the capsular polysaccharide-producing bacteria is a pathogenic bacteria.
72. The process of any one of paragraphs 1-70, wherein the capsular polysaccharide-producing bacteria is a pathogenic Streptococcus, a pathogenic Staphylococcus, a pathogenic Enterococcus, a pathogenic Bacillus, a pathogenic Corynebacterium, a pathogenic Listeria, a pathogenic Erysipelothrix, a pathogenic Clostridium, a pathogenic Haemophilus, a pathogenic Neisseria or a pathogenic Escherichia.
73. The process of any one of paragraphs 1-70, wherein the capsular polysaccharide-producing bacteria is a pathogenic Streptococcus, a pathogenic Staphylococcus or a pathogenic Enterococcus.
74. The process of any one of paragraphs 1-70, wherein the capsular polysaccharide-producing bacteria is Streptococcus pneumoniae, Streptococcus agalactiae, Streptococcus pyogenes, Staphylococcus aureus, Neisseria meningitidis, Escherichia coli, Salmonella typhi, Haemophilus influenzae, Klebsiella pneumoniae, Enterococcus faecium or Enterococcus faecalis. In an even preferred embodiment, the capsular polysaccharide-producing bacteria of the present invention is Streptococcus pneumoniae, Streptococcus agalactiae, or Streptococcus pyogenes.
75. The process of any one of paragraphs 1-70, wherein the capsular polysaccharide-producing bacteria is Streptococcus pneumoniae.
76. The process of any one of paragraphs 1-70, wherein the capsular polysaccharide-producing bacteria is Staphylococcus aureus.
77. The process of any one of paragraphs 1-70, wherein the capsular polysaccharide-producing bacteria is Enterococcus faecalis.
78. The process of any one of paragraphs 1-70, wherein the capsular polysaccharide-producing bacteria is Haemophilus influenzae type b.
79. The process of any one of paragraphs 1-70, wherein the capsular polysaccharide-producing bacteria is Neisseria meningitidis.
80. The process of any one of paragraphs 1-70, wherein the capsular polysaccharide-producing bacteria is Escherichia coli.
81. The process of any one of paragraphs 1-70, wherein the capsular polysaccharide-producing bacteria is Streptococcus agalactiae (Group B streptococcus (GBS)).
82. The process of any one of paragraphs 1-70, wherein the capsular polysaccharide-producing bacteria is Streptococcus agalactiae type Ia, Ib, II, III, IV, V, VI, VII, VIII or IX.
83. The process of any one of paragraphs 1-70, wherein the capsular polysaccharide-producing bacteria is Streptococcus agalactiae type Ia, Ib, II, III, IV, V, VI, VII or VIII.
84. The process of any one of paragraphs 1-70, wherein the capsular polysaccharide-producing bacteria is Streptococcus agalactiae type Ia, Ib, II, III, IV or V.
85. The process of any one of paragraphs 1-70, wherein the capsular polysaccharide-producing bacteria is Streptococcus agalactiae type Ia, Ib, II, III or V.
86. The process of any one of paragraphs 1-70, wherein the capsular polysaccharide-producing bacteria is an Escherichia coli part of the Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), or Escherichia coli—enteroinvasive (EIEC).
87. The process of any one of paragraphs 1-70, wherein the capsular polysaccharide-producing bacteria is an Uropathogenic Escherichia coli (UPEC).
88. The process of any one of paragraphs 1-70, wherein the capsular polysaccharide-producing bacteria is Streptococcus pneumoniae serotype 1, 2, 3, 4, 5, 6A, 6B, 6C, 6D, 6E, 6F, 6G, 6H, 7A, 7B, 7C, 7F, 8, 9A, 9L, 9V, 9N, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F or 48.
89. The process of any one of paragraphs 1-70, wherein the capsular polysaccharide-producing bacteria is Streptococcus pneumoniae serotype 1, 2, 3, 4, 5, 6A, 6B, 6C, 7C, 7F, 8, 9V, 9N, 10A, 10B, 11A, 12F, 14, 15A, 15B, 15C, 16F, 17F, 18C, 19A, 19F, 20A, 20B, 21, 22A, 22F, 23A, 23B, 23F, 24B, 24F, 27, 29, 31, 33B, 33F, 34, 35B, 35F, 38, 45 or 46.
90. The process of any one of paragraphs 1-70, wherein the capsular polysaccharide-producing bacteria is Streptococcus pneumoniae serotype 1, 2, 3, 4, 5, 6A, 6B, 7F, 8, 9V, 9N, 10A, 11A, 12F, 14, 15A, 15B, 15C, 16F, 17F, 18C, 19A, 19F, 20A, 20B, 22F, 23A, 23B, 23F, 24F, 27, 29, 31, 33F or 35B.
91. The process of any one of paragraphs 1-70, wherein the capsular polysaccharide-producing bacteria is Streptococcus pneumoniae serotype 1, 3, 4, 5, 6A, 6B, 7F, 8, 9V, 9N, 10A, 11A, 12F, 14, 15A, 15B, 15C, 18C, 19A, 19F, 22F, 23F, or 33F.
92. The process of any one of paragraphs 1-70, wherein the capsular polysaccharide-producing bacteria is Streptococcus pneumoniae serotype 1, 4, 5, 6A, 6B, 7F, 8, 9V, 9N, 10A, 11A, 12F, 14, 15A, 15B, 15C, 18C, 19A, 19F, 22F, 23F, or 33F.
93. The process of any one of paragraphs 1-92, wherein the saccharide bearing an azido group is a monosaccharide bearing an azido group, a di-saccharide bearing an azido group or a tri-saccharide bearing an azido group.
94. The process of any one of paragraphs 1-92, wherein the saccharide bearing an azido group is a monosaccharide bearing an azido group or a di-saccharide bearing an azido group.
95. The process of any one of paragraphs 1-92, wherein the saccharide bearing an azido group is a monosaccharide bearing an azido group.
96. The process of any one of paragraphs 1-92, wherein the saccharide bearing an azido group is a trisaccharide bearing an azido group.
97. The process of any one of paragraphs 1-92, wherein the saccharide bearing an azido group is derived from nigerotriose, derived from maltotriose, derived from melezitose, derived from maltotriulose, derived from raffinose or derived from kestose.
98. The process of any one of paragraphs 1-92, wherein the saccharide bearing an azido group is derived from raffinose.
99. The process of any one of paragraphs 1-92, wherein the saccharide bearing an azido group is a disaccharide bearing an azido group.
100. The process of any one of paragraphs 1-92, wherein the saccharide bearing an azido group is derived from sucrose, derived from maltose, derived from lactose, derived from lactulose or derived from trehalose.
101. The process of any one of paragraphs 1-92, wherein the saccharide bearing an azido group is derived from sucrose.
102. The process of any one of paragraphs 1-92, wherein the saccharide bearing an azido group is an amino monosaccharide bearing an azido group.
103. The process of any one of paragraphs 1-92, wherein the saccharide bearing an azido group is an amino hexose bearing an azido group.
104. The process of any one of paragraphs 1-92, wherein the saccharide bearing an azido group is derived from galactosamine or glucosamine.
105. The process of any one of paragraphs 1-92, wherein the saccharide bearing an azido group is derived from glucosamine.
106. The process of any one of paragraphs 1-92, wherein the saccharide bearing an azido group is an N-acetylated monosaccharide bearing an azido group.
107. The process of any one of paragraphs 1-92, wherein the saccharide bearing an azido group is a N-acetylated hexose bearing an azido group.
108. The process of any one of paragraphs 1-92, wherein the saccharide bearing an azido group is derived from N-acetylgalactosamine, N-acetylglucosamine or N-acetylmannosamine.
109. The process of any one of paragraphs 1-92, wherein the saccharide bearing an azido group is N-Azidoacetyl-galactosamine or N-Azidoacetyl-glucosamine.
110. The process of any one of paragraphs 1-92, wherein the saccharide bearing an azido group is N-Azidoacetyl-glucosamine.
111. The process of any one of paragraphs 1-92, wherein the saccharide bearing an azido group is an uronic acid bearing an azido group.
112. The process of any one of paragraphs 1-92, wherein the saccharide bearing an azido group is an hexuronic acid bearing an azido group.
113. The process of any one of paragraphs 1-92, wherein the saccharide bearing an azido group is derived from glucuronic acid or galacturonic acid.
114. The process of any one of paragraphs 1-92, wherein the saccharide bearing an azido group is derived from glucuronic acid or galacturonic acid and the azido group is located at position C2, C3 or C4.
115. The process of any one of paragraphs 1-92, wherein the saccharide bearing an azido group is derived from glucuronic acid or galacturonic acid and the azido group is located at position C2 or C3.
116. The process of any one of paragraphs 1-92, wherein the saccharide bearing an azido group is derived from glucuronic acid.
117. The process of any one of paragraphs 1-92, wherein the saccharide bearing an azido group is derived from glucuronic acid and the azido group is located at position C2 or C3.
118. The process of any one of paragraphs 1-92, wherein the saccharide bearing an azido group is a monosaccharide.
119. The process of any one of paragraphs 1-92, wherein the saccharide bearing an azido group is a monosaccharide derived from a triose, a tetrose, a pentose, an hexose or an heptose.
120. The process of any one of paragraphs 1-92, wherein the saccharide bearing an azido group is a monosaccharide derived from a pentose or an hexose.
121. The process of any one of paragraphs 1-92, wherein the saccharide bearing an azido group is a monosaccharide derived from an hexose.
122. The process of any one of paragraphs 1-92, wherein the saccharide bearing an azido group is a monosaccharide derived from a ketose.
123. The process of any one of paragraphs 1-92, wherein the saccharide bearing an azido group is a monosaccharide derived from an aldose.
124. The process of any one of paragraphs 1-92, wherein the saccharide bearing an azido group is derived from an aldopentose or an aldohexose.
125. The process of any one of paragraphs 1-92, wherein the saccharide bearing an azido group is derived from a pentose.
126. The process of any one of paragraphs 1-92, wherein the saccharide bearing an azido group is derived from arabinose, lyxose, ribose or xylose.
127. The process of any one of paragraphs 1-92, wherein the saccharide bearing an azido group is derived from arabinose or ribose.
128. The process of any one of paragraphs 1-92, wherein the saccharide bearing an azido group is derived from glucose, galactose or fructose.
129. The process of any one of paragraphs 1-92, wherein the saccharide bearing an azido group is derived from glucose or galactose.
130. The process of any one of paragraphs 1-92, wherein the saccharide bearing an azido group is derived from galactose.
131. The process of any one of paragraphs 1-92, wherein the saccharide bearing an azido group is derived from glucose.
132. The process of any one of paragraphs 1-92, wherein the saccharide bearing an azido group is derived from an hexose and the azido group is located at position C1, C2, C3, C4, C5 or C6.
133. The process of any one of paragraphs 1-92, wherein the saccharide bearing an azido group is derived from an hexose and the azido group is located at position C2, C3, C4 or C5.
134. The process of any one of paragraphs 1-92, wherein the saccharide bearing an azido group is derived from an hexose and the azido group is located at position C2, C3 or C4.
135. The process of any one of paragraphs 1-92, wherein the saccharide bearing an azido group is derived from an hexose and the azido group is located at position C2 or C3.
136. The process of any one of paragraphs 1-92, wherein the saccharide bearing an azido group is derived from an hexose and the azido group is located at position C3.
137. The process of any one of paragraphs 1-92, wherein the saccharide bearing an azido group is derived from an hexose and the azido group is located at position C2.
138. The process of any one of paragraphs 1-92, wherein the saccharide bearing an azido group is derived from galactose and the azido group is located at position C1, C2, C3, C4, C5 or C6.
139. The process of any one of paragraphs 1-92, wherein the saccharide bearing an azido group is derived from galactose and the azido group is located at position C2 or C3.
140. The process of any one of paragraphs 1-92, wherein the saccharide bearing an azido group is derived from galactose and the azido group is located at position C2.
141. The process of any one of paragraphs 1-92, wherein the saccharide bearing an azido is derived from glucose and the azido group is located at position C1, C2, C3, C4, C5 or C6.
142. The process of any one of paragraphs 1-92, wherein the saccharide bearing an azido group is derived from glucose and the azido group is located at position C2 or C3.
143. The process of any one of paragraphs 1-92, wherein the saccharide bearing an azido group is derived from glucose and the azido group is located at position C2.
144. The process of any one of paragraphs 1-143, wherein the saccharide bearing an azido group is a racemate.
145. The process of any one of paragraphs 1-143, wherein the saccharide bearing an azido group is a L-isomer.
146. The process of any one of paragraphs 1-143, wherein the saccharide bearing an azido group is a D-isomer.
147. The process of any one of paragraphs 1-92, wherein the saccharide bearing an azido group is derived from dextrose.
148. The process of any one of paragraphs 1-92, wherein the saccharide bearing an azido group is derived from dextrose and the azido group is located at position C2 or C3.
149. The process of any one of paragraphs 1-92, wherein the saccharide bearing an azido group is derived from dextrose and the azido group is located at position C2.
150. An isolated capsular polysaccharide comprising azido groups produced according to the process of any one of paragraphs 1-149.
151. The isolated capsular polysaccharide comprising azido groups of paragraph 150, comprising at least one azido groups for every 200 saccharide repeat units of the saccharide.
152. The isolated capsular polysaccharide comprising azido groups of paragraph 150, comprising at least one azido groups for every 100 saccharide repeat units of the saccharide.
153. The isolated capsular polysaccharide comprising azido groups of paragraph 150, comprising about one to ten azido groups for every 100 saccharide repeat units of the saccharide.
154. The isolated capsular polysaccharide comprising azido groups of paragraph 150, comprising about one to five azido groups for every 100 saccharide repeat units of the saccharide.
155. The isolated capsular polysaccharide comprising azido groups of any one of paragraphs 150-154, comprising no linker between the capsular polysaccharide and the azido groups.
156. The isolated capsular polysaccharide comprising azido groups of any one of paragraphs 150-155 which is a capsular saccharide from a pathogenic bacteria.
157. The isolated capsular polysaccharide comprising azido groups of any one of paragraphs 150-155 which is a capsular saccharide from a pathogenic Streptococcus, a pathogenic Staphylococcus, a pathogenic Enterococcus, a pathogenic Bacillus, a pathogenic Corynebacterium, a pathogenic Listeria, a pathogenic Erysipelothrix, a pathogenic Clostridium, a pathogenic Haemophilus, a pathogenic Neisseria or a pathogenic Escherichia.
158. The isolated capsular polysaccharide comprising azido groups of any one of paragraphs 150-155 which is a capsular saccharide from a pathogenic Streptococcus, a pathogenic Neisseria or a pathogenic Escherichia.
159. The isolated capsular polysaccharide comprising azido groups of any one of paragraphs 150-155 which is a capsular saccharide from Streptococcus pneumoniae, Streptococcus agalactiae, Streptococcus pyogenes, Staphylococcus aureus, Neisseria meningitidis, Escherichia coli, Salmonella typhi, Haemophilus influenzae, Klebsiella pneumoniae, Enterococcus faecium or Enterococcus faecalis.
160. The isolated capsular polysaccharide comprising azido groups of any one of paragraphs 150-155 which is a capsular saccharide from Streptococcus pneumoniae, Streptococcus agalactiae, or Streptococcus pyogenes.
161. The isolated capsular polysaccharide comprising azido groups of any one of paragraphs 150-155 which is a capsular saccharide from Streptococcus pneumoniae.
162. The isolated capsular polysaccharide comprising azido groups of any one of paragraphs 150-155 which is a capsular saccharide from Staphylococcus aureus.
163. The isolated capsular polysaccharide comprising azido groups of any one of paragraphs 150-155 which is a capsular saccharide from Enterococcus faecalis.
164. The isolated capsular polysaccharide comprising azido groups of any one of paragraphs 150-155 which is a capsular saccharide from Haemophilus influenzae type b.
165. The isolated capsular polysaccharide comprising azido groups of any one of paragraphs 150-155 which is a capsular saccharide from Neisseria meningitidis.
166. The isolated capsular polysaccharide comprising azido groups of any one of paragraphs 150-155 which is a capsular saccharide from Escherichia coli.
167. The isolated capsular polysaccharide comprising azido groups of any one of paragraphs 150-155 which is a capsular saccharide from Streptococcus agalactiae (Group B streptococcus (GBS)).
168. The isolated capsular polysaccharide comprising azido groups of any one of paragraphs 150-155 which is a capsular saccharide from Streptococcus agalactiae type Ia, Ib, II, III, IV, V, VI, VII, VIII or IX.
169. The isolated capsular polysaccharide comprising azido groups of any one of paragraphs 150-155 which is a capsular saccharide from Streptococcus agalactiae type Ia, Ib, II, III, IV, V, VI, VII or VIII.
170. The isolated capsular polysaccharide comprising azido groups of any one of paragraphs 150-155 which is a capsular saccharide from Streptococcus agalactiae type Ia, Ib, II, III, IV or V.
171. The isolated capsular polysaccharide comprising azido groups of any one of paragraphs 150-155 which is a capsular saccharide from Streptococcus agalactiae type Ia, Ib, II, III or V.
172. The isolated capsular polysaccharide comprising azido groups of any one of paragraphs 150-155 which is a capsular saccharide from Streptococcus pneumoniae serotype 1, 2, 3, 4, 5, 6A, 6B, 6C, 6D, 6E, 6F, 6G, 6H, 7A, 7B, 7C, 7F, 8, 9A, 9L, 9V, 9N, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F or 48.
173. The isolated capsular polysaccharide comprising azido groups of any one of paragraphs 150-155 which is a capsular saccharide from Streptococcus pneumoniae serotype 1, 2, 3, 4, 5, 6A, 6B, 6C, 7C, 7F, 8, 9V, 9N, 10A, 10B, 11A, 12F, 14, 15A, 15B, 15C, 16F, 17F, 18C, 19A, 19F, 20, 21, 22A, 22F, 23A, 23B, 23F, 24B, 24F, 27, 29, 31, 33B, 33F, 34, 35B, 35F, 38, 45 or 46.
174. The isolated capsular polysaccharide comprising azido groups of any one of paragraphs 150-155 which is a capsular saccharide from Streptococcus pneumoniae serotype 1, 2, 3, 4, 5, 6A, 6B, 7F, 8, 9V, 9N, 10A, 11A, 12F, 14, 15A, 15B, 15C, 16F, 17F, 18C, 19A, 19F, 20A, 20B, 22F, 23A, 23B, 23F, 24F, 27, 29, 31, 33F or 35B.
175. The isolated capsular polysaccharide comprising azido groups of any one of paragraphs 150-155 which is a capsular saccharide from Streptococcus pneumoniae serotype 1, 3, 4, 5, 6A, 6B, 7F, 8, 9V, 9N, 10A, 11A, 12F, 14, 15A, 15B, 15C, 18C, 19A, 19F, 22F, 23F, or 33F.
176. The isolated capsular polysaccharide comprising azido groups of any one of paragraphs 150-155 which is a capsular saccharide from Streptococcus pneumoniae serotype 1, 4, 5, 6A, 6B, 7F, 8, 9V, 9N, 10A, 11A, 12F, 14, 15A, 15B, 15C, 18C, 19A, 19F, 22F, 23F, or 33F.
177. The isolated capsular polysaccharide comprising azido groups of any one of paragraphs 150-176 which has a weight average molecular weight between 50 kDa and 5000 kDa.
178. The isolated capsular polysaccharide comprising azido groups of any one of paragraphs 150-176 which has a weight average molecular weight between 500 kDa and 5000 kDa.
179. The isolated capsular polysaccharide comprising azido groups of any one of paragraphs 150-176 which has a weight average molecular weight between 1000 kDa and 5000 kDa.
180. The isolated capsular polysaccharide comprising azido groups of any one of paragraphs 150-176 which is reduced by mechanical homogenization.
181. The isolated capsular polysaccharide comprising azido groups of any one of paragraphs 150-176 which is reduced by high pressure homogenization.
182. The isolated capsular polysaccharide comprising azido groups of any one of paragraphs 150-176 which is sized to a weight average molecular weight between 10 kDa and 1000 kDa.
183. The isolated capsular polysaccharide comprising azido groups of any one of paragraphs 150-176 which is sized to a weight average molecular weight between 50 kDa and 500 kDa.
184. The isolated capsular polysaccharide comprising azido groups of any one of paragraphs 150-176 which is sized to a weight average molecular weight between 50 kDa and 400 kDa.
185. The isolated capsular polysaccharide comprising azido groups of any one of paragraphs 150-176 which is sized to a weight average molecular weight between 100 kDa and 750 kDa.
186. The isolated capsular polysaccharide comprising azido groups of any one of paragraphs 150-176 which is sized to a weight average molecular weight between 100 kDa and 500 kDa.
187. The isolated capsular polysaccharide comprising azido groups of any one of paragraphs 150-176 which is not sized.
188. A method of making a glycoconjugate comprising the step of reacting the isolated capsular polysaccharide comprising azido groups of any one of paragraphs 150-187 with an alkyne functionalized carrier protein by Cu+1 mediated azide-alkyne cycloaddition reaction to form a glycoconjugate.
189. The method of paragraph 188 wherein said alkyne functionalized carrier protein is alkyne functionalized DT, TT, CRM₁₉₇, detoxified pneumococcal pneumolysin, PhtA, PhtB, PhtD, PhtE, OMPC, PorB, PD, PspA, PsaA or C5a peptidase from Streptococcus (SCP).
190. The method of paragraph 188 wherein said alkyne functionalized carrier protein is alkyne functionalized TT, DT, DT mutants (such as CRM₁₉₇) or a C5a peptidase from Streptococcus (SCP).
191. The method of paragraph 188 wherein said alkyne functionalized carrier protein is an alkyne functionalized DT (Diphtheria Toxoid).
192. The method of paragraph 188 wherein said alkyne functionalized carrier protein is an alkyne functionalized TT (Tetanus Toxoid).
193. The method of paragraph 188 wherein said alkyne functionalized carrier protein is alkyne functionalized PD (H. influenzae protein D).
194. The method of paragraph 188 wherein said alkyne functionalized carrier protein is alkyne functionalized CRM₁₉₇ or C5a peptidase from Streptococcus (SCP).
195. The method of paragraph 188 wherein said alkyne functionalized carrier protein is alkyne functionalized CRM₁₉₇.
196. The method of paragraph 188 wherein said an alkyne functionalized carrier protein is alkyne functionalized A chain of CRM₁₉₇.
197. The method of paragraph 188 wherein said alkyne functionalized carrier protein is alkyne functionalized SCP.
198. The method of any one of paragraphs 188-197, wherein said alkyne functionalized carrier protein bears a propargyl group.
199. The method of any one of paragraphs 188-198, wherein following step (a), unreacted azido groups in the conjugate, are capped using a suitable azido group capping agent.
200. The method of paragraph 199, wherein the azido group capping agent is an agent bearing an alkyne group.
201. The method of paragraph 199, wherein the azido group capping agent is an agent bearing a terminal alkyne.
202. The method of paragraph 199, wherein the azido group capping agent is an agent bearing a cycloalkyne.
203. The method of paragraph 199, wherein the azido group capping agent is propargyl alcohol.
204. The method of any one of paragraphs 188-203, wherein following step (a), the method further comprises a step of capping the unreacted alkyne groups remained in the conjugates with an alkyne group capping agent.
205. The method of paragraph 204, wherein said alkyne group capping agent is an agent bearing an azido group.
206. The method of paragraph 204, wherein said alkyne group capping agent is 3-azido-1-propanol.
207. A glycoconjugate produced according to the method of any one of paragraphs 188-206.
208. An immunogenic composition comprising a glycoconjugate of paragraph 207.

As used herein, the term “about” means within a statistically meaningful range of a value, such as a stated concentration range, time frame, molecular weight, temperature or pH. Such a range can be within an order of magnitude, typically within 20%, more typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every number within the range is also contemplated as an embodiment of the disclosure.

The terms “comprising”, “comprise” and “comprises” herein are intended by the inventors to be optionally substitutable with the terms “consisting essentially of”, “consist essentially of”, “consists essentially of”, “consisting of”, “consist of” and “consists of”, respectively, in every instance.

An “immunogenic amount”, an “immunologically effective amount”, a “therapeutically effective amount”, a “prophylactically effective amount”, or “dose”, each of which is used interchangeably herein, generally refers to the amount of antigen or immunogenic composition sufficient to elicit an immune response, either a cellular (T cell) or humoral (B cell or antibody) response, or both, as measured by standard assays known to one skilled in the art.

Any whole number integer within any of the ranges of the present document is contemplated as an embodiment of the disclosure.

All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

### EXAMPLES

### Example 1 Measurement of the Incorporation of Azide in Capsular Polysaccharide

A SEC fluorescence and Refractive index assay (SEC-FL/RI) was used for azide measurement to confirm the incorporation of azide modification.

Click-It Cell Reaction Buffer kit and Alexa Fluor 488 Alkyne were purchased from Invitrogen.

The initial procedure when running the SEC-FL/RI was to prepare the sample to a target concentration of 0.01 mg/m by mixing a pre-diluted polysaccharide to the final target concentration with the Click Reaction mixture. The reaction mixture consisted of 2.67 mM CuSO4, 2.67 mg/mL cell additive reagent, and 20 uM Alexa Fluor Alkyne. This was mixed with the sample at a 3:1 ratio of reaction mixture to sample and incubated in the absence of light. Reactions were transferred to HPLC vials and ran on the SEC-FL/RI HPLC setup. The RI detector (RID) allowed for detection of the polysaccharide while the Fluorescence Detector (FLD) was used to determine the level of fluor incorporated into the polysaccharide sample. The ratio of the two peak areas gave a measurement for the amount of azide incorporated to the polysaccharide.

This methodology required targeting a constant polysaccharide concentration in order for the ratio to be comparable across different runs. Later, in addition to the incubation of the sample for thirty minutes, a buffer exchange step was added that washed out the unreacted fluor as well as the reaction matrix reagents in order to improve the baselines of the FLD and RID during the HPLC run. This added greater resolution of the peak associated with the azide functionalized polysaccharide. The assay was useful for relative quantitation of azide incorporation into the polysaccharide but could not quantify an exact numerical concentration. To improve this aspect work was undertaken to use a polysaccharide with known levels of azido functionalization to make a standard curve that varied the % Azido while keeping the polysaccharide mass the same. This was done by mixing the known azido modified poly (polysaccharide) with specific amounts of native (unmodified) polysaccharide. A strong linear fit for the generated standard curve was seen.

The values for percent azide were able to be converted to a μM azide per mM polysaccharide by converting from % azide using the constant polysaccharide concentration.

Table 1 captures the conversion of the standard curve from % azide to μM azide per mM polysaccharide.

TABLE 1

| % Azido Poly | % Azide | μM Azide/mM Poly | FL/RI |
|---|---|---|---|
| 0 | 0.00 | 0.00 | 0.73 |
| 20 | 0.18 | 1.80 | 110.23 |
| 40 | 0.36 | 3.60 | 256.45 |
| 60 | 0.54 | 5.40 | 461.84 |
| 80 | 0.72 | 7.20 | 681.06 |
| 100 | 0.90 | 9.00 | 945.59 |

### Example 2 Media formulation

Composition of the media used for seed expansion (seed expansion media) is provided at table 2.

TABLE 2
Seed expansion media

Concentration

| Component | g/L | mL/L |
|---|---|---|
| R17 Dry Powder * | 19.45 | — |
| L-Tyrosine Disodium Salt, Dihydrate | 0.8 | — |
| Dextrose, Anhydrous ** | 24 | — |
| Sodium Chloride | 1.38 | — |
| Acidic Cysteine Solution (300 mM) | — | 4.69 |
| L-Asparagine, Monohydrate | 2.81 | — |
| L-Glutamine | 1.46 | — |
| Ferrous Sulfate Solution (1 mM) | — | 6.25 |
| Trace E Elements Solution | — | 1.25 |
| Magnesium Sulfate, Heptahydrate | 1.32 | — |
| MOPS (200 mM, pH 8) | — | 200 |

* For R17 composition, see e.g. WO2017085602 Table 4 p. 35
** concentration may vary in the experiments, and dextrose may be replaced with Galactose in some of the experiments, see details below.

The production medium is similar to the seed expansion media but did not contain MOPS. The pH was adjusted to 7.33 (or other pH see details below) using 5N NAOH.

The Anthrone assay was used to measure polysaccharide concentration by reaction of the polysaccharide to the anthrone reagent.

### Example 3 Azido-incorporation into S. pneumoniae Serotype 14 (T14) Capsular Polysaccharide (Poly) During Fermentation, Azido Sugar Screening

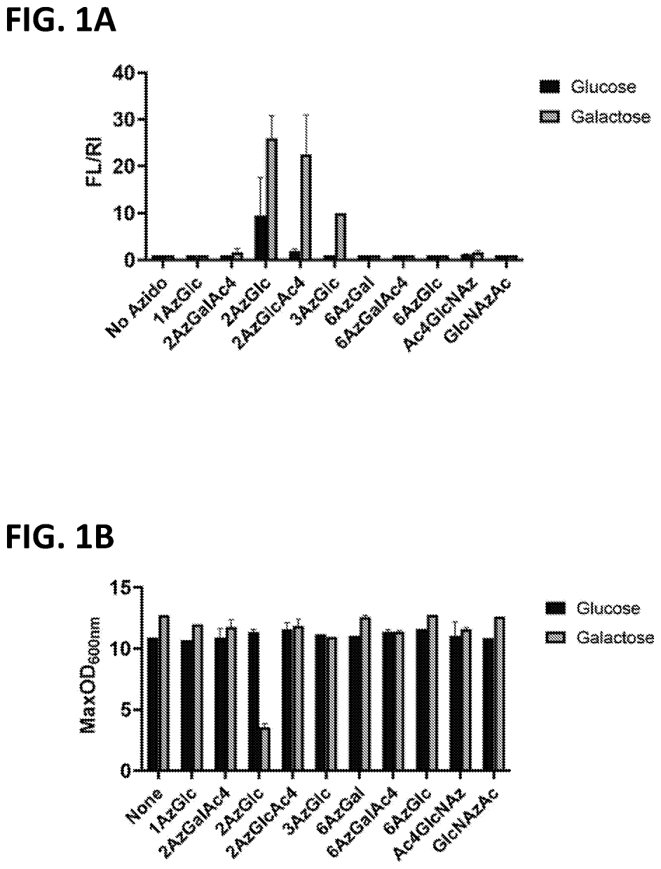

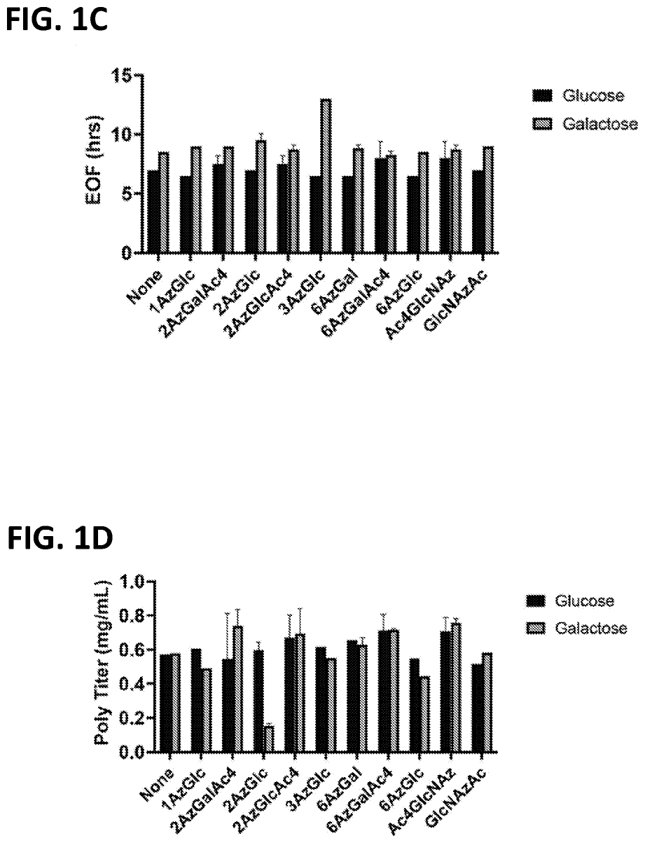

To identify azido sugars that could incorporate into S. pneumoniae T14 capsule, we grew S. pneumoniae T14 (serotype 14) in the production medium supplemented with various azido sugars (Table 3) supplied at 1 mM. We performed our screen using both production medium supplemented with Glucose (dextrose, 30 g/L) and production medium supplemented with Galactose (30 g/L). We identified three azido sugars (2AzGlc, 2AzGlcAc4, and 3AzGlc) that were incorporated into T14 capsule at detectable levels (i.e. FL/RI was above 1.0) (FIG. 1A). The growth substrate affected the incorporation of all three azido sugars. 2AzGlc, 2AzGlcAc4, and 3AzGlc were all detectable in polysaccharide from bacteria grown in production medium supplemented with Galactose, but only 2AzGlc produced a detectable FL/RI when the bacteria were grown in production medium supplemented with Glucose. Additionally, growth and polysaccharide production were affected by azido sugar supplementation, but only for azido sugars that produced detectable FL/RI (FIGS. 11B-1D). The impact of azido sugars on growth and polysaccharide production was dependent on the growth sugar: 2AzGlc reduced the maximum OD and polysaccharide titer and 3AzGlc elongated the End Of Fermentation (EOF) time but only in production medium supplemented with Galactose.

TABLE 3
Azido Sugars Utilized

| Azido Sugar Name | Shorthand |
|---|---|
| β-D-glucopyranosyl azido | 1AzGlc |
| 2-Azido-2-deoxy-D-glucose | 2AzGlc |
| 3-azido-3-deoxy-D-glucopyranose | 3AzGlc |
| 6-azido-6-deoxy-D-glucopyranose | 6AzGlc |
| 2-azido-2-deoxy-D-galactose | 2AzGal |
| 6-azido-6-deoxy-D-galactose | 6AzGal |
| N-azidoacetyl-glucosamine | GlcNAcAz |
| 3,4-Di-O-acetyl-2-azido-2-deoxy-D-glucopyranose | 2AzGlcA2 |
| 3,6-Di-O-acetyl-2-azido-2-deoxy-D-glucopyranose | 2AzGlcAc2 |
| 1,3,4,6-Tetra-O-acetyl-2-azido-2-deoxy-D- | 2AzGlcAc4 |
| glucopyranose | |
| 1,2,4,6-Tetra-O-acetyl-3-azido-glucopyranose | 3AzGlcAc4 |
| 1,3,4,6-Tetra-O-acetyl-2-azido-2-deoxy-D- | 2AzGalAc4 |
| galactopyranose | |
| 1,2,3,4-Tetra-O-acetyl-6-azido-6-deoxy-D- | 6AzGalAc4 |
| galactopyranose | |
| 1,3,4,6-Tetra-O-acetyl-N-azidoacetylglucosamine | Ac4GlcNAcAz |

### Example 4 Azido-Incorporation into S. pneumoniae Serotype 14 (T14) Capsular Polysaccharide (Poly) During Fermentation, 2AzGlc Titration

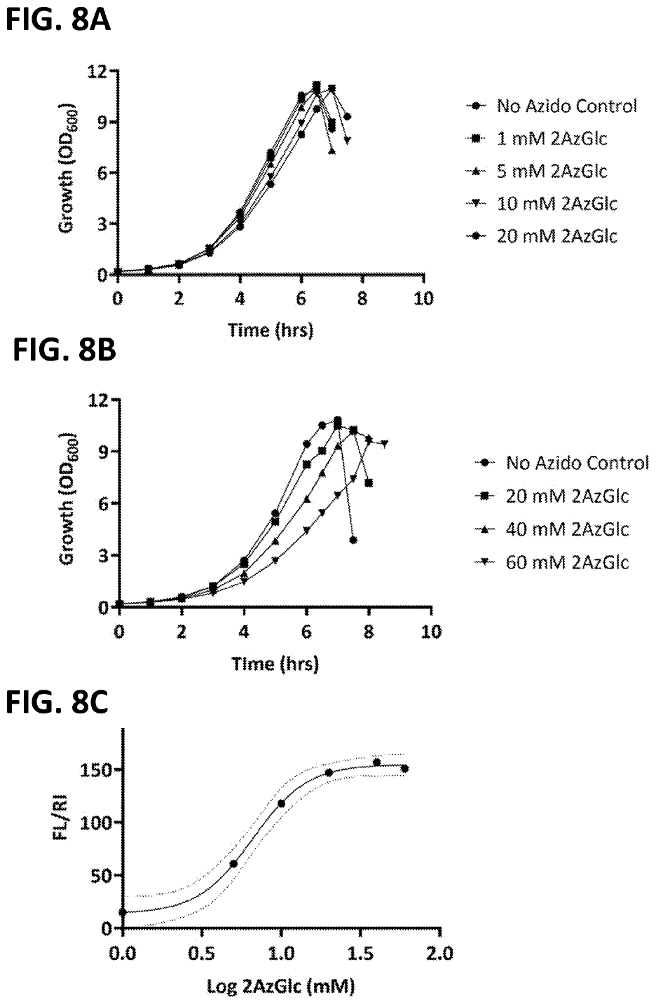

We tested the impact of 2AzGlc concentration on S. pneumoniae T14 growth, polysaccharide production, and azido incorporation by growing the bacteria in production media (supplemented with Glucose (dextrose, 30 g/L)) with various concentrations of 2AzGlc (0, 1, 5, 10, 20, 40, and 60 mM). 2AzGlc concentration was negatively associated with growth, negatively associated with poly production, but positively associated with azido incorporation into T14 capsule polysaccharide (FIGS. 8A-8C and Table 4). The negative effect of 2AzGlc on growth was small and concentration dependent, started at 20 mM, and generated the following phenotypes: longer total fermentation time, reduction in maximum OD600 nm, and a decrease in the rate of autolysis. The negative effect of 2AzGlc on poly production was more pronounced (max titer loss=56%, max size reduction=78%), started at a much lower concentration (1 mM), but plateaued at 20 mM 2AzGlc. The positive effect of 2AzGlc concentration on FL/RI was also robust (10-fold increase between lowest and higher FL/RI achieved), was also detectable at low concentrations (1 mM) and plateaued around 20 mM.

TABLE 4
Impact of 2AzGlc Concentration on T14
Poly Production and Azido Incorporation

| Media pH | 2AzGlc (mM) | Titer (mg/mL) | Size (kDa) | FL/RI |
|---|---|---|---|---|
| 7 | 0 | 0.57 | 1158 | <1 |
| | 1 | 0.63 | 674 | 15 |
| | 5 | 0.53 | 430 | 61 |
| | 10 | 0.43 | 310 | 118 |
| | 20 | 0.32 | 280 | 146 |
| 7.33 | 0 | 0.63 | 1197 | <1 |
| | 20 | 0.3 | 865 | 148 |
| | 40 | 0.28 | 765 | 157 |
| | 60 | 0.28 | 753 | 151 |

### Example 5 Azido-Incorporation into S. pneumoniae Serotype 14 (T14) Capsular Polysaccharide (Poly) During Fermentation, pH and Agitation DOE

To improve the incorporation of 2AzGlc into S. pneumoniae T14 capsule in production media (supplemented with Glucose (dextrose, 30 g/L)), we performed a 16-vessel DOE designed to test the impact of pH (6.33, 6.66, 7.00, and 7.33) and agitation (100, 200, and 300 rpm) on four response variables (MaxOD, Poly Titer, Poly Size, and FL/RI). Seed expansion media (supplemented with Glucose (dextrose, 24 g/L)) was used for seed expansion and production media (supplemented with Galactose (30 g/L))+2AzGlc (10 mM) was used for the DOE. The fermentations were performed in 100 ml working volume vessels, using a 10-fold dilution of the seed bottle culture (OD600 nm=1-1.2) for the inoculum, a temperature setpoint of 36° C., and an air overlay of 1.5 SLPH. The pH was controlled during fermentation. The raw data collected for this DOE (Table 5) was analyzed using the JMP14 software package via a least squares regression model emphasizing effect screening.

Our analysis shows that both pH and agitation significantly affect the model, which is predictive for 3 of the 4 response variables (MaxOD, poly titer, and FL/RI). pH significantly impacts both growth and FL/RI while agitation significantly impacts poly titer. There is no evidence that pH and agitation are confounded. Importantly, the model is strongly predictive for incorporation and showed that the highest FL/RI values could be achieved using a pH setpoint of 7.33 and agitation between 100-200 rpm.

TABLE 5
Data Input and Output for pH/Agitation Model Development

| | | | | Poly | Poly | |
|---|---|---|---|---|---|---|
| | | Agitation | MaxOD600 | Titer | Size | FL/ |
| Vessel | pH | (rpm) | nm | (mg/mL) | (kDa) | RI |
| 1 | 6.66 | 200 | 11.7 | 0.47 | 450 | 135.0 |
| 2 | 7.33 | 100 | 9.6 | 0.37 | 646 | 158.3 |
| 3 | 6.33 | 300 | 10.7 | 0.42 | 606 | 97.5 |
| 4 | 7 | 300 | 11.3 | 0.43 | 689 | 115.7 |
| 5 | 7.33 | 100 | 8.9 | 0.36 | 794 | 137.4 |
| 6 | 7 | 100 | 8.8 | 0.40 | 788 | 117.8 |
| 7 | 6.33 | 100 | 12.1 | 0.39 | 723 | 91.9 |
| 8 | 6.33 | 100 | 13.6 | 0.39 | 566 | 100.7 |
| 9 | 7.33 | 300 | 10.5 | 0.41 | 693 | 113.9 |
| 10 | 7.33 | 300 | 10.4 | 0.41 | 720 | 137.3 |
| 11 | 6.33 | 300 | 10.8 | 0.44 | 597 | 98.3 |
| 12 | 7.33 | 200 | 8.5 | 0.41 | 678 | 140.8 |
| 13 | 6.66 | 200 | 11.7 | 0.52 | 552 | 114.5 |
| 14 | 7 | 100 | 7.8 | 0.41 | 752 | 118.8 |
| 15 | 7 | 300 | 12.9 | 0.48 | 639 | 107.5 |
| 16 | 7.33 | 200 | 8.3 | 0.44 | 781 | 125.0 |

### Example 6 Azido Incorporation Studies

To understand the different mechanisms that could influence azido sugar incorporation into bacterial capsule requires some knowledge of bacterial metabolism, regulation, and biosynthesis of capsule polysaccharide.

### 1 Capsule Biosynthesis

Capsule biosynthesis involves five major steps: 1) import of monosaccharides 2) synthesis of nucleotide-monosaccharides 3) repeat unit formation, 4) flipping of repeat units across the bacterial membrane, and 5) capsule chain elongation. The ideal azido monosaccharide needs to be compatible with these steps.

2 Sugar transport

S. pneumoniae has evolved to catabolize complex carbohydrates found in respiratory mucins and expresses a set of 30 different sugar transporters to import mono- and polysaccharides. Most bacterial sugar transporter fall into two categories, 1) ATP binding cassette (ABC)-transporters and 2) phosphotransferase system (PTS)-transporters. Both transporters use high energy phosphate bonds to power the transport of sugars across the bacterial membrane, but PTS-transporters also phosphorylate the imported sugar (typically occurs at carbon 6 for hexoses). Thus, in addition to affecting the affinity of any transporter for its sugar substrate, azido modification can block transport if 1) the transporter is a PTS transporter and 2) azido modification is on the carbon that accepts the phosphate group.

### 3 Carbon Catabolite Repression

Most bacteria exhibit a preference for specific sugars. The process by which the preferred downregulates the transport and/or catabolism of non-preferred sugars or substrates is called carbon catabolite repression (CCR). S. pneumoniae exhibits a preference for glucose over other sugars. Thus, glucose provided as the primary growth substrate may interfere with the uptake of non-glucose azido-sugars. Additionally, activation of CCR regulation can upregulate glycolysis, the primary catabolic pathway for sugars (i.e. azido sugars may be lost to glycolysis).

### 4 Nucleotide Sugar Formation

Biosynthesis of nucleotide sugars involves the entry of sugars into central metabolism and their conversion into the different nucleotide sugars. Azido modification may inhibit sugar phosphorylation (the first step in entry into central metabolism) and/or lower the affinity of pathway enzymes for their sugar substrate. Affecting enzyme affinity can be good or bad, depending on whether the enzyme belongs to a catabolic pathway (e.g. glycolysis) or a nucleotide-sugar forming pathway. Another consideration is abundance of each sugar molecule in the capsule of S. pneumoniae serotypes. While azido sugars can be interconverted once they become intracellular, the fewest number of process steps is most likely to result successful incorporation. Component analysis of each capsule serotype shows that while no individual sugar is present in all S. pneumoniae serotypes, glucose and galactose are both highly abundant and present in most capsule serotypes (Table 6).

TABLE 6
Monosaccharide Content of S. pneumoniae Capsules by Serotype

| | Sugars | | | | | | | | | | | | | | | |
|---|---|---|---|---|---|---|---|---|---|---|---|---|---|---|---|---|
| Sero- | per | | | | | | Rib- | Glyc- | | | | | | | | |
| type | Repeat | Glc | GlcA | Gal | GalA | Rha | ol | erol | GlcNAc | GalNAc | ManNAc | ManNAc | PneNAc | FucNAc | AATGal | Sug |
| 1 | 3 | | | | 2 | | | | | | | | | | 1 | |
| 3 | 2 | 1 | 1 | | | | | | | | | | | | | |
| 4 | 4 | | | 1 | | | | | | 1 | 1 | | | 1 | | |
| 5 | 5 | 1 | 1 | | | | | | | | | | 1 | 1 | | 1 |
| 6A | 4 | 1 | | 1 | | 1 | 1 | | | | | | | | | |
| 6B | 4 | 1 | | 1 | | 1 | 1 | | | | | | | | | |
| 7F | 7 | 1 | | 2 | | 2 | | | 1 | 1 | | | | | | |
| 8 | 4 | 2 | 1 | 1 | | | | | | | | | | | | |
| 9V | 5 | 2 | 1 | 1 | | | | | | | 1 | | | | | |
| 10A | 7 | | | 5 | | | 1 | | | 1 | | | | | | |
| 11A | 5 | 2 | | 2 | | | | 1 | | | | | | | | |
| 12F | 6 | 2 | | 1 | | | | | | 1 | | 1 | | 1 | | |
| 14 | 4 | 1 | | 2 | | | | | 1 | | | | | | | |
| 15B | 6 | 1 | | 3 | | | | 1 | 1 | | | | | | | |
| 18C | 6 | 3 | | 1 | | 1 | | 1 | | | | | | | | |
| 19A | 3 | 1 | | | | 1 | | | | | 1 | | | | | |
| 19F | 3 | 1 | | | | 1 | | | | | 1 | | | | | |
| 22F | 6 | 2 | 1 | 1 | | 2 | | | | | | | | | | |
| 23F | 5 | 1 | | 1 | | 2 | | 1 | | | | | | | | |
| 33F | 6 | | | 6 | | | | | | | | | | | | |

### 5 Sugar Polymerization

Azido modification has the potential to disrupt sugar polymerization by decreasing the affinity of the glycosyltransferases for the sugar substrate and/or blocking the site of sugar linkages. We therefore identified the bonding positions (C1, C2 . . . C6) for each monosaccharide present in each repeat unit for each serotype (this includes acetyl groups) (Table 7). We found C2, C5, and C6 were the carbons with the fewest linkages. C6 modification can limit the uptake and/or entry of hexoses into central metabolism, because this is the site of phosphorylation, making this a poor candidate site for azido modification. Thus, the most promising azido modification sites are C2 and C5.

TABLE 7
Intermolecular Bonding Positions of
S. pneumoniae Capsule Hexoses

# of Bonds Per Carbon Position
Per Capsule Repeat

| Serotype | C1 | C2 | C3 | C4 | C5 | C6 |
|---|---|---|---|---|---|---|
| 1 | 3 | 1 | 3 | 1 | | |
| 3 | 2 | | 1 | 1 | | |
| 4 | 4 | | 3 | 1 | | |
| 5 | 5 | 1 | 2 | 2 | | |
| 6A | 3 | 1 | 3 | | 1 | |
| 6B | 3 | 1 | 2 | 1 | 1 | |
| 7F | 7 | 2 | 2 | 4 | | 1 |
| 8 | 4 | | | 4 | | |
| 9V | 5 | | 2 | 3 | | |
| 10A | 6 | 1 | 3 | 1 | 2 | 1 |
| 11A | 5 | | 1 | 3 | | 1 |
| 12F | 6 | 1 | 3 | 2 | | |
| 14 | 4 | | 1 | 2 | | 1 |
| 15B | 5 | 2 | 2 | 2 | | 1 |
| 18C | 6 | 1 | 2 | 3 | | |
| 19A | 3 | | 1 | 2 | | |
| 19F | 3 | 1 | | 2 | | |
| 22F | 6 | 1 | 2 | 3 | | |
| 23F | 4 | 2 | 1 | 3 | | |
| 33F | 6 | 1 | 4 | | 1 | |

### Example 7 2AzGlcAc4 and 2AzGlc Titration

To further examine incorporation of 2AzGlc and 2AzGlcAc4 into S. pneumonia polysaccharide, we performed growth curves of the bacteria in production medium supplemented with Galactose (30 g/L) with a range of 2AzGlcAc4 and 2AzGlc concentrations.

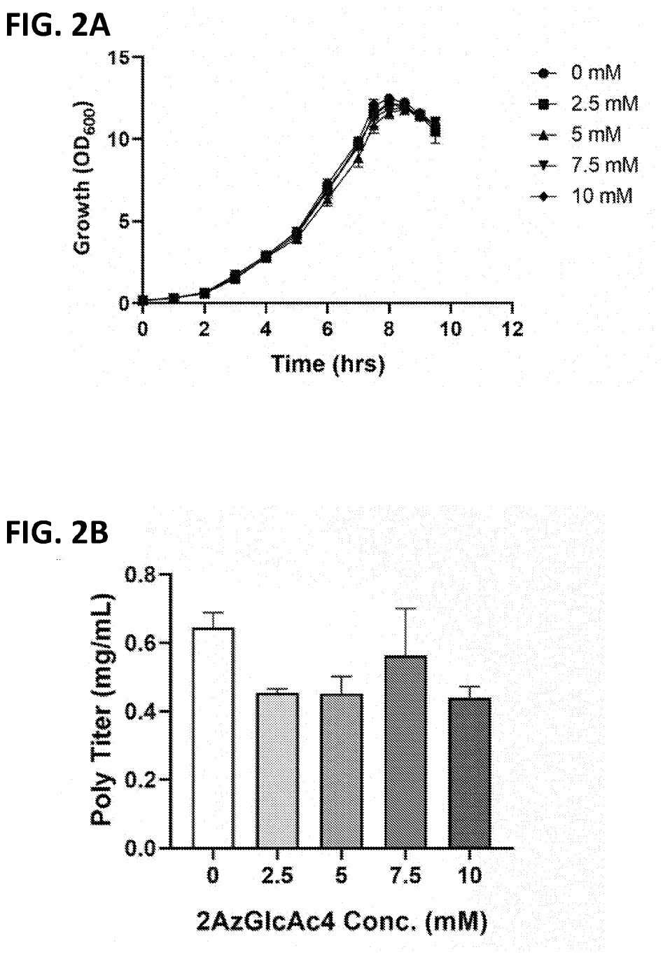

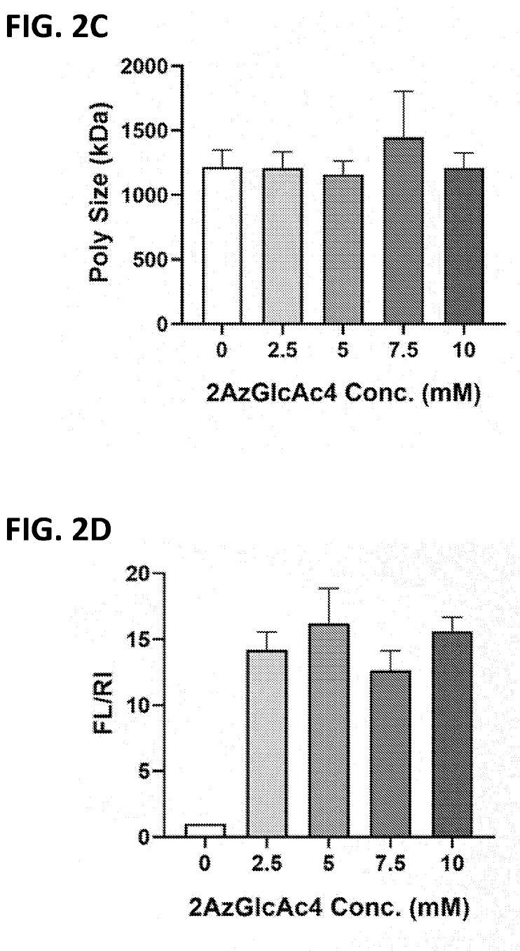

We tested 2AzGlcAc4 at 0, 2.5, 5, 7.5, and 10 mM. We observed no impact on growth or polysaccharide size (FIG. 2A-2C). However, all 2AzGlcAc4 azido concentrations caused a statistically significant reduction in polysaccharide titer, except 7.5 mM 2AzGlc (n=2) which had a higher standard deviation and fewer n than the rest of the conditions (FIG. 2B). The reduction in polysaccharide titer is likely caused by the addition of DMSO (2% final conc.) to the culture, not the azido sugar (DMSO has been shown to negatively affected polysaccharide production, in particular polysaccharide titer, data not shown). DMSO concentrations were high for this experiment as 2AzGlcAc4 was found to be highly insoluble in production medium supplemented with Galactose (30 g/L) (limit of solubility≤1.0 mM). It was therefore unsurprising that increasing concentrations of 2AzGlcAc4, all of which exceeded 1.0 mM, did not cause an increase in FL/RI and did not produce an FL/RI signal greater than that observed in our screen (FIG. 2D).

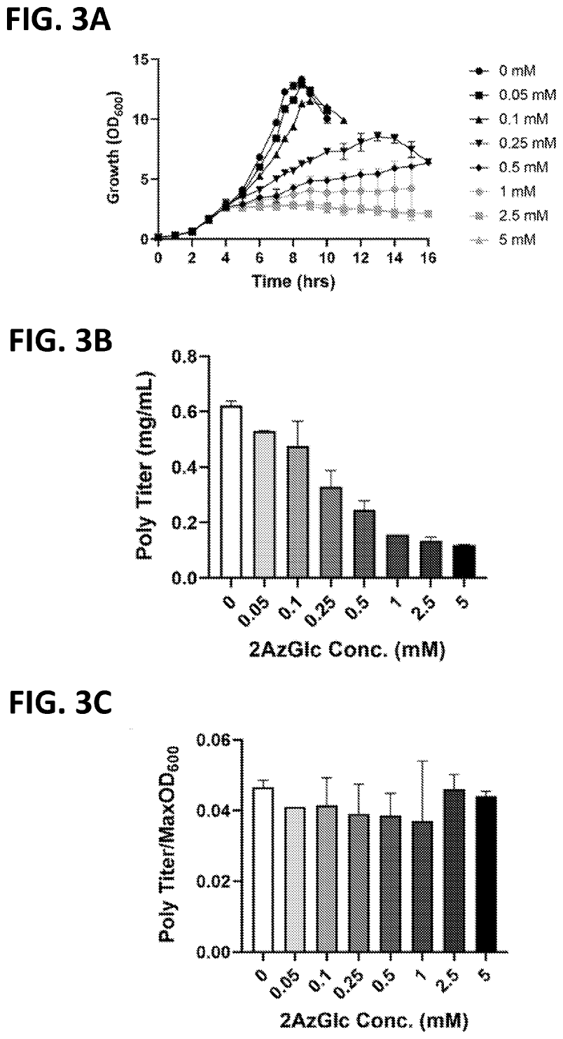

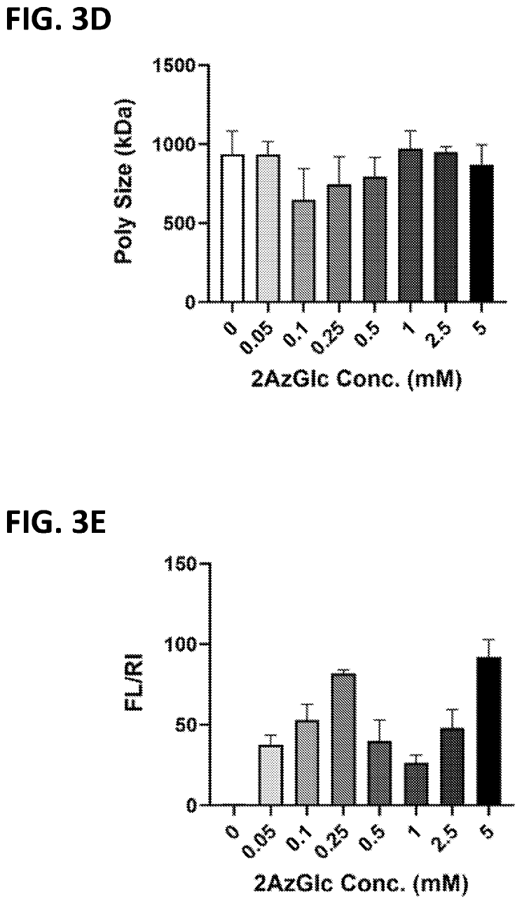

The results were very different for 2AzGlc (version that is not acylated), where we observed 1) much higher levels of incorporation and 2) a 2AzGlc concentration-dependent effect on growth, polysaccharide production, and incorporation. Growth and polysaccharide titers were negatively impacted by increasing 2AzGlc concentrations in a titratable fashion starting at the lowest 2AzGlc concentration provided (FIGS. 3A and 3B).

The ratio of the polysaccharide titer concentrations to maximum OD600 for each azido concentration are very similar; therefore, the impact of azido supplementation of polysaccharide titer is most likely to be indirect via growth inhibition (FIG. 3C). There was no observable effect of 2AzGlc concentration on polysaccharide size (FIG. 3D). The effect of 2AzGlc concentration on incorporation (i.e. FL/RI) was polynomial, with the highest levels of incorporation observed at 0.25 mM and 5 mM 2AzGlc (FIG. 3E). These data suggest that the 2AzGlc concentration is directly and positively related to incorporation.

### Example 8 Impact of Azido Sugar Acetylation

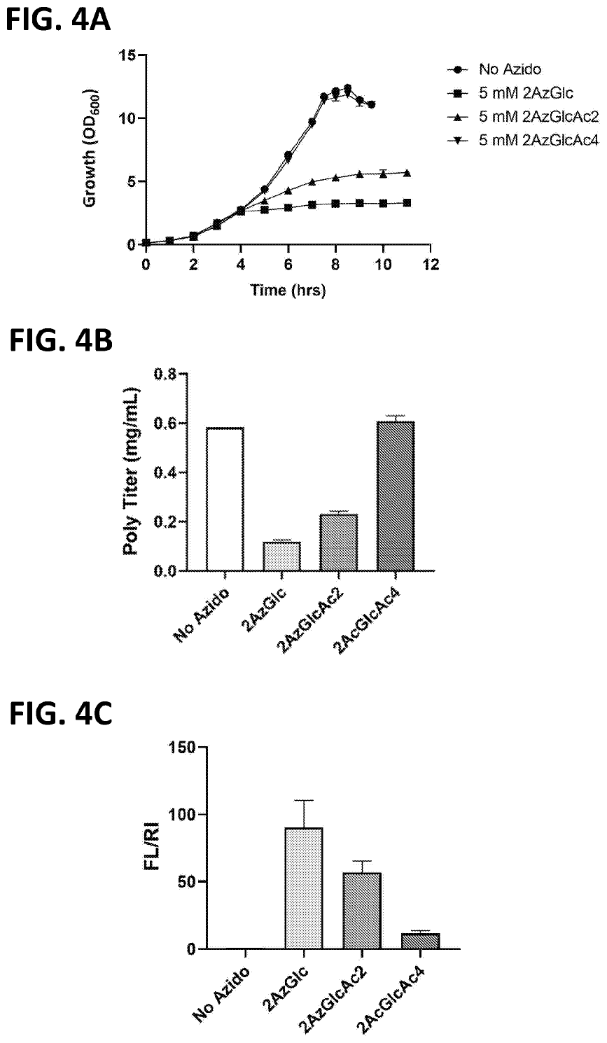

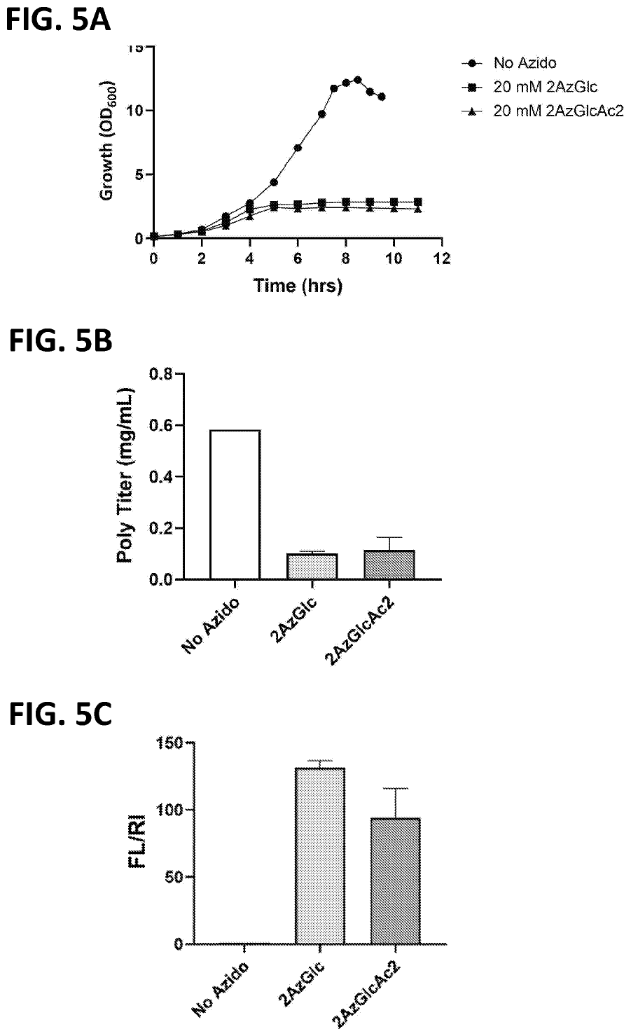

We examined the impact of acetylation on azido sugar incorporation into S. pneumoniae T14 capsule polysaccharide by growing the bacteria in production medium supplemented with Galactose (30 g/L) with un-, di-, and tetra-acetylated 2AzGlc. At 5 mM with 1% DMSO, we found a positive association between azido sugar acetylation and growth and azido sugar acetylation and poly titer (FIGS. 4A and 4B). However, we observed a negative association between azido sugar acetylation and azido incorporation (FL/RI) (FIG. 4C). When compared at high concentrations (20 mM, 2AzGlc and 2AzGlcAc2 only) without DMSO, there was no discernable association between azido sugar acetylation and growth or polysaccharide production (both were strongly inhibited) (FIGS. 5A and 5B). Despite this, the negative association between azido sugar acetylation and incorporation remained (FIG. 5C). These associations were further confirmed using 3AzGlc and 3AzGlcAc4 (data not shown).

### Example 9 pH and Agitation DOE (Design of Experiments)

We performed a 12-vessel DOE designed to test the impact of pH and agitation on four response variables (MaxOD, Poly Titer, Poly Size, and FL/RI). Seed expansion media supplemented with Galactose (30 g/L) was used for seed expansion and production medium supplemented with Galactose (30 g/L)+2AzGlc (0.25 mM) was used for the DOE. The fermentations were performed in 100 ml working volume vessels using a 10-fold dilution of the seed bottle culture (OD600 nm=1.0-1.2) for the inoculum, a temperature setpoint of 36° C., and an air overlay of 1.5 SLPH. The raw data collected for this DOE (Table 8) was analyzed using the JMP14 software package via a least squares regression model emphasizing effect screening. Our analysis shows that the model is predictive for 3 of the 4 response variables (MaxOD, poly titer, and FL/RI). The analysis shows that pH has a significant (p-value<0.00001) and large impact (LogWorth=6.3) while agitation has a significant (p-value=0.04) but very small (LogWorth<2) impact on the model. pH and agitation are confounded for FL/RI. Finally, the model shows that a maximum FL/RI is achieved at 300 rpm with a pH of 7.33.

TABLE 8
Data Input and Output for pH/Agitation DOE

| | | | Polysacch. | Polysacch. | |
|---|---|---|---|---|---|
| | Agitation | | Titer | Size | |
| pH | (rpm) | MaxOD | (mg/mL) | (kDa) | FL/RI |
| 7.33 | 300 | 8.26 | 0.211 | 1367 | 110.12 |
| 7.33 | 100 | 8.23 | 0.216 | 849 | 88.76 |
| 6.33 | 100 | 6.76 | 0.193 | 837 | 50.26 |
| 7 | 200 | 9.38 | 0.269 | 1134 | 97.25 |
| 6.33 | 100 | 6.98 | 0.195 | 781 | 51.73 |
| 6.66 | 100 | 8.96 | 0.247 | 982 | 78.29 |
| 7.33 | 300 | 8.54 | 0.228 | 1036 | 109.76 |
| 6.66 | 300 | 9.1 | 0.227 | 1658 | 77.94 |
| 7.33 | 100 | 8.43 | 0.235 | 784 | 101.26 |
| 6.33 | 300 | 6.05 | 0.087 | 659 | 45.24 |
| 6.33 | 300 | 5.89 | 0.149 | 718 | 44.39 |
| 6.33 | 200 | 6.81 | 0.171 | 783 | 48.41 |

### Example 10 Galactose Fed-Batch Fermentation with Azido Sugar

1 Fed Batch fermentation

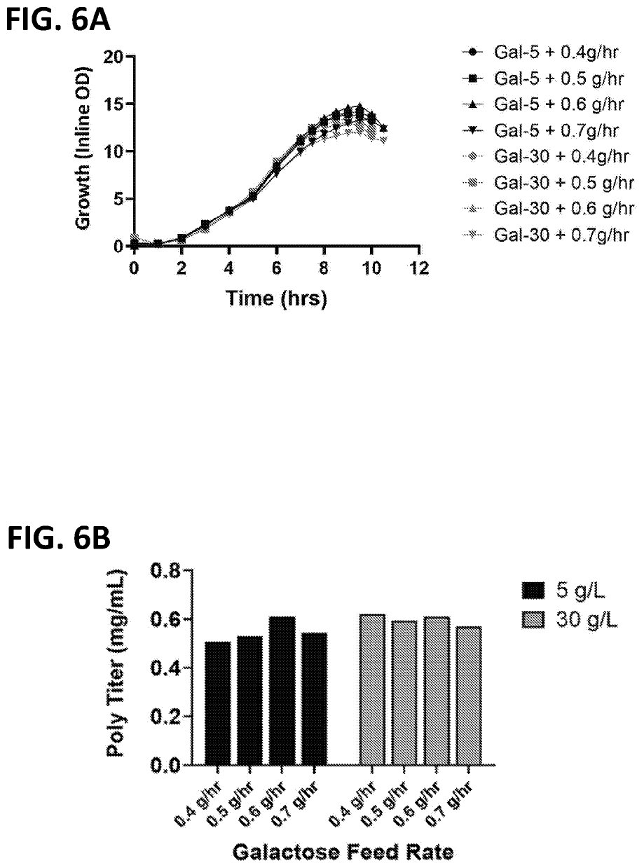

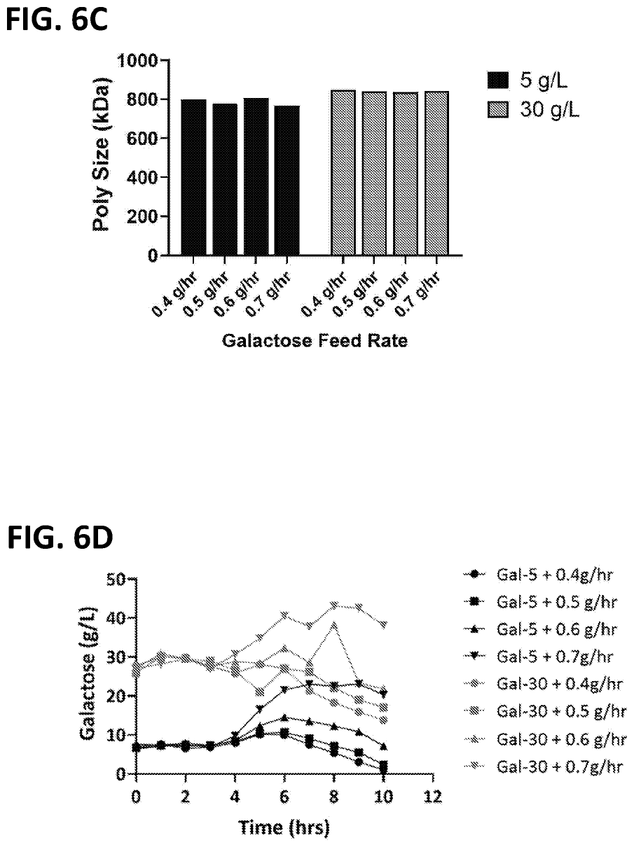

We developed fed-batch fermentation in order to test whether the growth sugar concentration affects the incorporation of azido monosaccharides. To do this, we grew S. pneumoniae in production medium with an initial galactose concentration of 5.0 g/L (low sugar) or 30.0 g/L (high sugar) and then supplied additional galactose as a feed from 3.5-8.5 hrs (Table 9). The data shows that there was no effect of lowering the galactose concentration from 30 to 5 g/L on growth but there was a slight reduction in polysaccharide titer and size (FIGS. 6A-6C).

Additionally, we found that the 0.5 and 0.6 g/hr galactose feeds were the best at maintaining the initial galactose media concentrations and that the feed rate did not affect growth or polysaccharide production except at 0.7 g/hr where it slightly reduced the growth rate of vessels with both 5.0 and 30.0 g/L galactose (FIGS. 6A-6D).

TABLE 9
Feed Rate Specifications for the S. pneumoniae
T14 Galactose Fed-Batch fermentation

| | Initial Gal | Galactose Feed | | | |
|---|---|---|---|---|---|
| | Conc. | Stock Conc. | Flow Rate | Feed | Feed Interval |
| Vessel | (g/L) | (g/L) | (mls/hr) | (g/hr) | (hr) |
| 1 | 5.0 | 285 | 1.4 | 0.4 | 3.5-8.5 |
| 2 | | 357 | | 0.5 | |
| 3 | | 429 | | 0.6 | |
| 4 | | 482 | | 0.7 | |
| 5 | 30.0 | 285 | | 0.4 | |
| 6 | | 357 | | 0.5 | |
| 7 | | 429 | | 0.6 | |
| 8 | | 482 | | 0.7 | |

2 Fed-Batch Fermentation with Azido Sugar

To test whether the concentration of the growth sugar in the media affects azido sugar incorporation and/or toxicity, we compared growth, polysaccharide production, and incorporation of 2AzGlc into S. pneumoniae T14 grown in batch fermentation (in production medium supplemented with Galactose (30 g/L)) to those grown in fed-batch fermentation (in production medium with an initial galactose concentration of 5.0 g/L). S. pneumoniae T14 was grown overnight in seed expansion media supplemented with Galactose (30 g/L) at 36° C. without shaking until the OD600 nm=1.0-1.2. The seed bottle culture was then diluted 10-fold into vessels (100 mL working volume) containing 90 mls of production media with either 5.0 or 30.0 g/L of galactose+/−0.25 mM 2AzGlc. All vessels had a single Rushton impeller, were agitated at 173 rpm, had an air overlay of 1.5 SLPH, were set to 36° C., had a pH setpoint of 7.0, and used 20% sodium carbonate for based control. Because 2AzGlc addition inhibits growth of S. pneumoniae T14 in production medium supplemented with Galactose (30 g/L) the rate of galactose consumption is lower (˜70% of the batch model). Anticipating that this would also happen at 5.0 g/L of galactose, we utilized two galactose feed rates for this study (galactose stock was 400 g/L) (Table 10). The first feed rate (FR #1) matches the galactose consumption of cells grown in production medium supplemented with Galactose (5 g/L) without azido addition. The second feed rate (FR #2) provides galactose at ˜70% of FR #1, but the feed is provided for an extended time interval.

TABLE 10
Feed Rate Specifications for the S. pneumoniae
T14 Galactose Fed-Batch fermentation

| Galactose Feed Rate #1 | | Galactose Feed Rate #2 | |
|---|---|---|---|
| HH:MM:SS | Flow Rate (mL/hr) | HH:MM:SS | Flow Rate (mL/hr) |
| 0:00:00 | 0 | 0:00:00 | 0 |
| 3:29:59 | 0 | 3:29:59 | 0 |
| 3:30:00 | 0.5 | 3:30:00 | 0.35 |
| 4:59:00 | 0.5 | 4:59:00 | 0.35 |
| 5:00:00 | 1.125 | 5:00:00 | 0.79 |
| 5:59:59 | 1.125 | 5:59:59 | 0.79 |
| 6:00:00 | 1.625 | 6:00:00 | 1.14 |
| 6:59:00 | 1.625 | 7:59:59 | 1.14 |
| 7:00:00 | 1.75 | 8:00:00 | 1.23 |
| 9:29:59 | 1.75 | 13:29:59 | 1.23 |
| 9:30:00 | 0 | 13:30:00 | 0 |

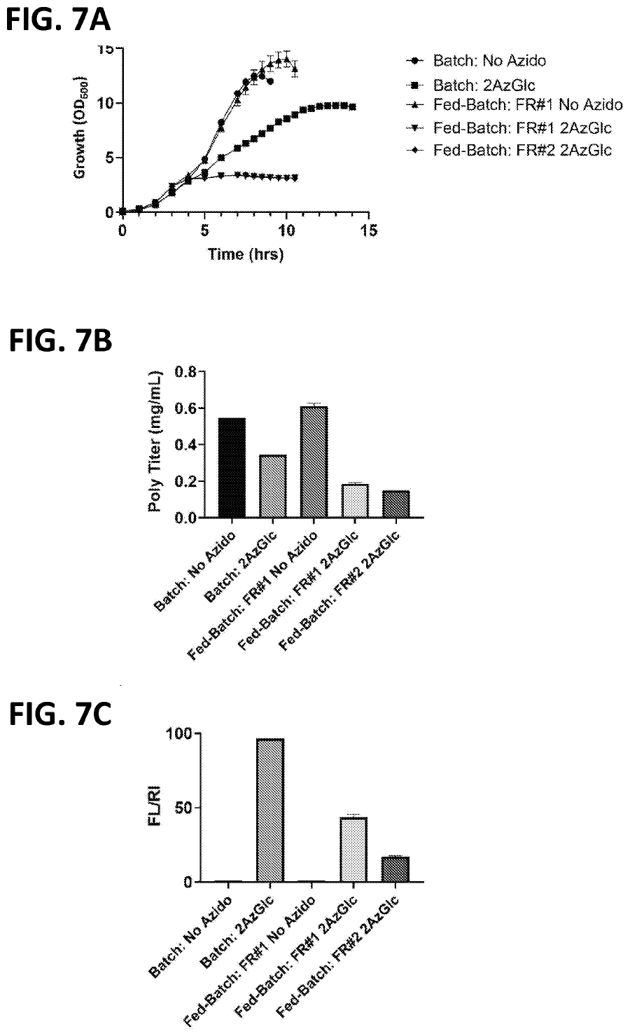

The results show that maintaining low media galactose levels decreases incorporation of 2AzGlc in S. pneumoniae T14 capsule polysaccharide (FIG. 70). Both batch controls (production medium supplemented with Galactose (30 g/L)+/−0.25 mM 2AzGlc) grew as expected, produced polysaccharide as expected, and had the expected levels of azido incorporation (i.e. FL/RI) (FIGS. 7A-7C). The no azido fed-batch controls (production medium supplemented with Galactose (5 g/L), FR #1) grew very similarly to the no azido batch control (production medium supplemented with Galactose (30 g/L)) but had a higher maximum OD600 nm and slightly longer total fermentation time. This suggests that either 1) 30 g/L of galactose is growth limiting (FR #1 supplies 3.15 g of galactose on top of the 0.5 g galactose present in the media to begin with, for a total of 3.65 g or 36.5 g/L) or 2) reduced galactose levels allow for more efficient growth. Growth of the fed-batch cultures with 0.25 mM 2AzGlc stopped at an OD600 nm=3.0, regardless of the feed rate. As a result, the polysaccharide titers were low. The two feed rates had a differential effect on azido incorporation, with FR #1 generating an FL/RI (45) that was over 2-fold higher than FR #2 FL/RI (18).

### 3 Azido Feed DOE

We performed a 16-vessel DOE to test the impact of elevating the initial galactose concentration (decreases the initial ratio of 2AzGlc:galactose), providing the azido sugar as a feed (further DOE details provided in Table 11), and having two different initial azido concentration (0 and 0.1 mM) on four response variables (MaxOD, Polysaccharide Titer, Polysaccharide Size, and FL/RI).

TABLE 11
Azido Feed DOE Design

| Input Factors | Values |
|---|---|
| Initial Galactose Conc. (g/L) | 30, 40 |
| Initial 2AzGlc Conc. (mM) | 0, 0.1 |
| 2AzGlc Feed Stock Conc. (mM) | 4.0, 6.5 |

Response Variables
MaxOD600
Poly Titer (mg/mL)
Poly Size (kDa)
FL/RI

Seed expansion media supplemented with Galactose was used for seed expansion and production medium media with either 30.0 or 40.0 g/L of galactose was used for the DOE. Fermenters (100 mL working volume) were inoculated with a 10-fold dilution of seed bottle culture (OD600 nm=1.0-1.2) and incubated at 36° C., at 300 rpm, with an air overlay of 1.5 SLPH, pH was controlled at 7.33.

2AzGlc was dissolved in WFI, sterilized, and then provided as a feed to each culture in a linearly increasing fashion (2AzGlc Feed Flow Rate (mIs/hr) was Y=0.02*X+0.4, where X=hr and time frame was 0-13 hrs). To generate different 2AzGlc feed rates we used two different stock concentrations (4 and 6.5 mM) with a matching feed rate. Additionally, the vessels had two different initial 2AzGlc concentrations (0 and 0.1 mM). Least squares regression analysis of raw DOE data (Table 12) shows that the all three input variables (initial galactose concentration, initial 2AzGlc concentration, and the 2AzGlc feed stock concentration) significantly affect the model and that the model is predicative for 3 of the 4 response variables (MaxOD600, Polysaccharide Titer, and FL/RI). However, the effect of the initial galactose concentration is very small (LogWorth<2) whereas the effect of the azido feed concentration and initial azido media concentration is much greater (LogWorth>2). Azido sugar concentration (initial 2AzGlc conc. and 2AzGlc stock conc.) was significant for MaxOD, polysaccharide titer, and FL/RI whereas initial galactose levels were only significant for polysaccharide titer. The model predicts that the maximum FL/RI is achieved using an initial galactose level of 40 g/L, an initial 2AzGlc concentration of 0.1 mM, and a 4.0 mM 2AzGlc feed stock. Using these conditions, we achieved an average FL/RI of 123.7, polysaccharide titer of 0.1815 mg/mL, and a maximum OD600 nm of 8.5. This is an increase in FL/RI over the 110 FL/RI value achieved during the pH and agitation DOE for the batch fermentation (see Table 8).

TABLE 12
DOE Raw Data Table

| Gal | Initial | 2AzGlc | | | | |
|---|---|---|---|---|---|---|
| Conc. | 2AzGlc | Stock | | Poly | Poly | |
| (g/L) | Conc. (mM) | Conc. (mM) | MaxOD | Titer | Size | FL/RI |
| 30 | 0.1 | 6.5 | 5.65 | 0.137 | 1319 | 83.69 |
| 30 | 0 | 4 | 9.56 | 0.22 | 1357 | 103.61 |
| 40 | 0.1 | 4 | 8.4 | 0.1748 | 1306 | 122.41 |
| 30 | 0.1 | 4 | 8.96 | 0.1856 | 1265 | 116.16 |
| 40 | 0 | 4 | 9.78 | 0.2204 | 1317 | 97.04 |
| 40 | 0.1 | 4 | 8.53 | 0.1882 | 1188 | 125.02 |
| 40 | 0 | 6.5 | 8.44 | 0.1826 | 1385 | 122.7 |
| 30 | 0 | 6.5 | 8.74 | 0.1846 | 1440 | 113.82 |
| 40 | 0 | 4 | 7.39 | 0.1796 | 1088 | 114.22 |
| 30 | 0.1 | 4 | 8.13 | 0.1876 | 1272 | 116.31 |
| 40 | 0.1 | 6.5 | 4.53 | 0.1251 | 1363 | 79.3 |
| 40 | 0 | 6.5 | 6.64 | 0.1536 | 1190 | 110.09 |
| 40 | 0.1 | 6.5 | 3.71 | 0.1011 | 1318 | 63.19 |
| 30 | 0 | 4 | 9.78 | 0.2358 | 1079 | 101.45 |
| 30 | 0 | 6.5 | 8.32 | 0.189 | 951 | 121.9 |
| 30 | 0.1 | 6.5 | 5.76 | 0.1266 | 1220 | 84.53 |

1. A process for producing a bacterial capsular polysaccharide comprising azido groups, comprising the step of cultivating a capsular polysaccharide-producing bacteria by fermentation in a cell culture media supplemented with a saccharide bearing an azido group.

2. The process of claim 1 comprising the step of:
(i) inoculating a first container containing a cell culture medium with seed stock of the bacteria, and incubating the first container until growth requirements are met, and
(ii) inoculating a second container containing a cell culture media supplemented with a saccharide bearing an azido group with the culture from step (i), and incubating said second container until growth requirements are met.

3. The process of claim 1 which further comprises the step of purifying the bacterial capsular polysaccharide comprising azido groups.

4. The process of claim 1 comprising the step of:
(a) cultivating a capsular polysaccharide-producing bacteria in a cell culture medium, which includes: (i) inoculating a first container containing a cell culture medium with seed stock of the bacteria, and incubating the first container until growth requirements are met, (ii) inoculating a second container containing a cell culture media supplemented with a saccharide bearing an azido group with the culture from step (i), and incubating said second container until growth requirements are met,
(b) purifying the bacterial capsular polysaccharide from said bacteria, thereby obtaining a bacterial capsular polysaccharide comprising azido groups.

5. The process of any one of claims 1-4, wherein the culture in said cell culture media supplemented with a saccharide bearing an azido group is batch culture, fed batch culture or continuous culture.

6. The process of any one of claims 1-5, wherein the culture in said cell culture media supplemented with a saccharide bearing an azido group last until the culture reaches an OD₆₀₀ of between about 4 and about 100.

7. The process of any one of claims 1-5, wherein the culture in said cell culture media supplemented with a saccharide bearing an azido group last until the ΔOD600≤0 over a period of at least 30 minutes.

8. The process of any one of claims 1-7, wherein the cell culture media supplemented with a saccharide bearing an azido group comprises between 5 mM and 60 mM of saccharide bearing an azido group.

9. The process of any one of claims 1-8, wherein the cell culture media supplemented with a saccharide bearing an azido group further comprises a growth saccharide.

10. The process of claim 9, wherein said growth saccharide is a monosaccharide or an oligosaccharide.

11. The process of claim 9, wherein said growth saccharide is glucose or galactose.

12. The process of any one of claims 9-11, wherein the growth saccharide is similar to the saccharide bearing an azido group used to supplement the cell culture media.

13. The process of any one of claims 1-12, wherein the capsular polysaccharide-producing bacteria is a pathogenic bacteria.

14. The process of any one of claims 1-12, wherein the capsular polysaccharide-producing bacteria is a pathogenic Streptococcus, a pathogenic Staphylococcus, a pathogenic Enterococcus, a pathogenic Bacillus, a pathogenic Corynebacterium, a pathogenic Listeria, a pathogenic Erysipelothrix, a pathogenic Clostridium, a pathogenic Haemophilus, a pathogenic Neisseria or a pathogenic Escherichia.

15. The process of any one of claims 1-12, wherein the capsular polysaccharide-producing bacteria is Streptococcus pneumoniae, Streptococcus agalactiae, or Streptococcus pyogenes.

16. The process of any one of claims 1-12, wherein the capsular polysaccharide-producing bacteria is Streptococcus pneumoniae serotype 1, 2, 3, 4, 5, 6A, 6B, 6C, 6D, 6E, 6F, 6G, 6H, 7A, 7B, 7C, 7F, 8, 9A, 9L, 9V, 9N, 10A, 10B, 10C, 10F, 11A, 11B, 11C, 11D, 11E, 11F, 12A, 12B, 12F, 13, 14, 15A, 15B, 15C, 15F, 16A, 16F, 17A, 17F, 18A, 18B, 18C, 18F, 19A, 19B, 19C, 19F, 20A, 20B, 21, 22A, 22F, 23A, 23B, 23F, 24A, 24B, 24F, 25A, 25F, 27, 28A, 28F, 29, 31, 32A, 32F, 33A, 33B, 33C, 33D, 33E, 33F, 34, 35A, 35B, 35C, 35F, 36, 37, 38, 39, 40, 41A, 41F, 42, 43, 44, 45, 46, 47A, 47F or 48.

17. The process of any one of claims 1-16, wherein the saccharide bearing an azido group is a monosaccharide bearing an azido group, a di-saccharide bearing an azido group or a tri-saccharide bearing an azido group.

18. The process of any one of claims 1-16, wherein the saccharide bearing an azido group is an amino monosaccharide bearing an azido group, a N-acetylated monosaccharide bearing an azido group, N-Azidoacetyl-galactosamine, N-Azidoacetyl-glucosamine or an uronic acid bearing an azido group.

19. The process of any one of claims 1-16, wherein the saccharide bearing an azido group is a monosaccharide derived from an hexose.

20. The process of any one of claims 1-16, wherein the saccharide bearing an azido group is derived from glucose or galactose.

21. The process of any one of claims 1-16, wherein the saccharide bearing an azido group is derived from an hexose and the azido group is located at position C2, C3, C4 or C5.

22. The process of any one of claims 1-16, wherein the saccharide bearing an azido group is derived from galactose and the azido group is located at position C2 or C3.

23. The process of any one of claims 1-16, wherein the saccharide bearing an azido group is derived from glucose and the azido group is located at position C2 or C3.

24. An isolated capsular polysaccharide comprising azido groups produced according to the process of any one of claims 1-23.

25. The isolated capsular polysaccharide comprising azido groups of claim 24, comprising at least one azido groups for every 200 saccharide repeat units of the saccharide.

26. The isolated capsular polysaccharide comprising azido groups of claim 24, comprising about one to ten azido groups for every 100 saccharide repeat units of the saccharide.

27. The isolated capsular polysaccharide comprising azido groups of any one of claims 24-26, comprising no linker between the capsular polysaccharide and the azido groups.

28. A method of making a glycoconjugate comprising the step of reacting the isolated capsular polysaccharide comprising azido groups of any one of claims 24-26 with an alkyne functionalized carrier protein by Cu+1 mediated azide-alkyne cycloaddition reaction to form a glycoconjugate.

29. The method of claim 28 wherein said alkyne functionalized carrier protein is alkyne functionalized DT, TT, CRM₁₉₇, detoxified pneumococcal pneumolysin, PhtA, PhtB, PhtD, PhtE, OMPC, PorB, PD, PspA, PsaA or C5a peptidase from Streptococcus (SCP).

30. The method of any one of claims 28-29, wherein said alkyne functionalized carrier protein bears a propargyl group.

31. A glycoconjugate produced according to the method of any one of claims 28-30.

32. An immunogenic composition comprising a glycoconjugate of claim 31.
